(19) United States
(12) Patent Application Publication
(10) Pub. No.: US 2011/0184178 A1
(43) Pub. Date: Jul. 28, 2011

(54) POLYMORPHS OF 5-(3-(ETHYLSULFONYL)PHENYL)-3,8-DIMETHYL-N-(1-METHYLPIPERIDIN-4-YL)-9H-PYRIDO[2,3-B]INDOLE-7-CARBOXAMIDE AND METHODS OF USE THEREFOR

(75) Inventors: Paul Isbester, Castleton-on-Hudson, NY (US); Bingidimi I. Mobele, Altamont, NY (US); Grant J. Palmer, Nashville, TN (US); Jonathon S. Salsbury, Madison, WI (US); Luckner Ulysse, Albany, NY (US)

(73) Assignee: Takeda Pharmaceutical Company Limited, Osaka-shi, Osaka (JP)

(21) Appl. No.: 12/988,627

(22) PCT Filed: Apr. 14, 2009
(86) PCT No.: PCT/US09/40556

§ 371 Date: Apr. 5, 2011

Related U.S. Application Data
(60) Provisional application No. 61/045,458, filed on Apr. 16, 2008.

(51) Int. Cl. C07D 471/04

(52) U.S. Cl. 546/194

(57) ABSTRACT

The invention relates to polymorphic forms of 5-(3-(ethylsulfonyl)phenyl)-3,8-dimethyl-N-(1-methylpiperidin-4-yl)-9h-pyrido[2,3-b]indole-7-carboxamide (referred to herein as Compound I), which has the formula:

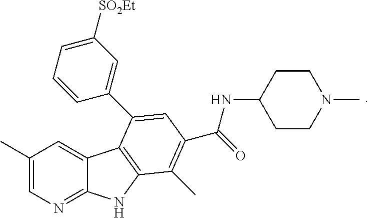

The invention also relates to compositions thereof and methods for the preparation of the polymorphs of Compound I, and kits and articles of manufacture of the compositions, and methods of using the compositions to treat various diseases.

### FIELD OF THE INVENTION

The present invention relates generally to polymorphic forms of 5-(3-(ethylsulfonyl)phenyl)-3,8-dimethyl-N-(1-methylpiperidin-4-yl)-9H-pyrido[2,3-b]indole-7-carboxamide, (referred to herein as “Compound I”) and methods for their preparation. It also related to salts of Compound I. The present invention also relates to pharmaceutical compositions, kits and articles of manufacture comprising polymorphs of Compound I, and methods of their use.

### DESCRIPTION OF RELATED ART

Compound I, which has the formula:

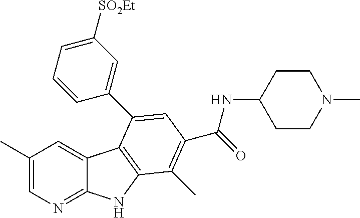

is a kinase inhibitor that is described in U.S. Patent Publication No. 2007-0117816, published May 24, 2007 (see Compound 112) and U.S. Patent Application Nos. 60/912,625 and 60/912,629, filed Apr. 18, 2007 (see Compound 83), which are incorporated herein by reference in their entireties.

Phosphoryl transferases are a large family of enzymes that transfer phosphorous-containing groups from one substrate to another. By the conventions set forth by the Nomenclature Committee of the International Union of Biochemistry and Molecular Biology (IUBMB) enzymes of this type have Enzyme Commission (EC) numbers starting with 2.7. - . - (See, Bairoch A., The ENZYME database in Nucleic Acids Res. 28:204-305 (2000)). Kinases are a class of enzymes that function in the catalysis of phosphoryl transfer. The protein kinases constitute the largest subfamily of structurally related phosphoryl transferases and are responsible for the control of a wide variety of signal transduction processes within the cell. (See, Hardie, G. and Hanks, S. (1995) The Protein Kinase Facts Book, I and II, Academic Press, San Diego, Calif.). Protein kinases are thought to have evolved from a common ancestral gene due to the conservation of their structure and catalytic function. Almost all kinases contain a similar 250-300 amino acid catalytic domain. The protein kinases may be categorized into families by the substrates they phosphorylate (e.g., protein-tyrosine, protein-serine/threonine, histidine, etc.). Protein kinase sequence motifs have been identified that generally correspond to each of these kinase families (See, for example, Hanks, S. K.; Hunter, T., FASEB J. 9:576-596 (1995); Kinghton et al., Science, 253:407-414 (1991); Hiles et al., Cell 70:419-429 (1992); Kunz et al., Cell, 73:585-596 (1993); Garcia-Bustos et al., EMBO J., 13:2352-2361 (1994)). Lipid kinases (e.g. PI3K) constitute a separate group of kinases with structural similarity to protein kinases.

Protein and lipid kinases regulate many different cell processes including, but not limited to, proliferation, growth, differentiation, metabolism, cell cycle events, apoptosis, motility, transcription, translation and other signaling processes, by adding phosphate groups to targets such as proteins or lipids. Phosphorylation events catalyzed by kinases act as molecular on/off switches that can modulate or regulate the biological function of the target protein. Phosphorylation of target proteins occurs in response to a variety of extracellular signals (hormones, neurotransmitters, growth and differentiation factors, etc.), cell cycle events, environmental or nutritional stresses, etc. Protein and lipid kinases can function in signaling pathways to activate or inactivate, or modulate the activity of (either directly or indirectly) the targets. These targets may include, for example, metabolic enzymes, regulatory proteins, receptors, cytoskeletal proteins, ion channels or pumps, or transcription factors. Uncontrolled signaling due to defective control of protein phosphorylation has been implicated in a number of diseases and disease conditions, including, for example, inflammation, cancer, allergy/asthma, diseases and conditions of the immune system, disease and conditions of the central nervous system (CNS), cardiovascular disease, dermatology, and angiogenesis.

Initial interest in protein kinases as pharmacological targets was stimulated by the findings that many viral oncogenes encode structurally modified cellular protein kinases with constitutive enzyme activity. These findings pointed to the potential involvement of oncogene related protein kinases in human proliferatives disorders. Subsequently, deregulated protein kinase activity, resulting from a variety of more subtle mechanisms, has been implicated in the pathophysiology of a number of important human disorders including, for example, cancer, CNS conditions, and immunologically related diseases. The development of selective protein kinase inhibitors that can block the disease pathologies and/or symptoms resulting from aberrant protein kinase activity has therefore generated much interest.

Cancer results from the deregulation of the normal processes that control cell division, differentiation and apoptotic cell death. Protein kinases play a critical role in this regulatory process. A partial non-limiting list of such kinases includes abl, Aurora-A, Aurora-B, Aurora-C, ATK, bcr-abl, Blk, Brk, Btk, c-Kit, c-Met, c-Src, CDK1, CDK2, CDK4, CDK6, cRafl, CSF1R, CSK, EGFR, ErbB2, ErbB3, ErbB4, ERK, Fak, fes, FGFR1, FGFR2, FGFR3, FGFR4, FGFR5, Fgr, FLK-4, Flt-1, Fps, Frk, Fyn, Hck, IGF-1R, INS—R, Jak, KDR, Lck, Lyn, MEK, p38, PDGFR, PIK, PKC, PYK2, Ros, Tie1, Tie2, Trk, Yes and Zap70. In mammalian biology, such protein kinases comprise mitogen activated protein kinase (MAPK) signaling pathways. MAPK signaling pathways are inappropriately activated by a variety of common disease-associated mechanisms such as mutation of ras genes and deregulation of growth factor receptors (Magnuson et al., Seminars in Cancer Biology 5:247-252 (1994)). Therefore the inhibition of protein kinases is an object of the present invention.

Aurora kinases (Aurora-A, Aurora-B, Aurora-C) are serine/threonine protein kinases that have been implicated in human cancer, such as colon, breast and other solid tumors. Aurora-A (also sometimes referred to as AIK) is believed to be involved in protein phosphorylation events that regulate the cell cycle. Specifically, Aurora-A may play a role in controlling the accurate segregation of chromosomes during mitosis. Misregulation of the cell cycle can lead to cellular proliferation and other abnormalities. In human colon cancer tissue, Aurora-A, Aurora-B and Aurora-C have been found to be overexpressed (See, Bischoff et al., EMBO J., 17:3052-3065 (1998); Schumacher et al., J. Cell Biol. 143:1635-1646 (1998); Kimura et al., J. Biol. Chem., 272:13766-13771 (1997)).

Kinase inhibitors are believed to be useful agents for the prevention, delay of progression, and/or treatment of conditions mediated by kinases.

### SUMMARY OF THE INVENTION

The present invention provides novel polymorphic forms of Compound I and methods of preparing these polymorphic forms, as well as compositions comprising one or more of the novel polymorphs.

### Polymorphic Forms

In one aspect, the invention provides polymorphic forms of the free base form of Compound I having the formula:

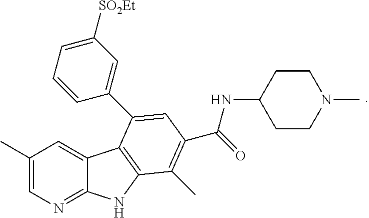

Various methods are also provided for making Form A, Form B, Form C, Form D, Form E, Form F and Form G. Various methods are also provided for manufacturing pharmaceutical compositions, kits and other articles of manufacture comprising one or more of Form A, Form B, Form C, Form D, Form E, Form F and Form G.

### Form A:

In one embodiment, the polymorphic form has an X-ray powder diffraction pattern (CuKα) comprising significant diffraction peaks at about 5.4, 17.3 and 20.2 degrees 2-theta (° 2θ). In some variations, the X-ray powder diffraction pattern further comprises significant diffraction peaks at about 16.7, 20.7 and 25.2 °2θ. In other variations, the X-ray diffraction pattern is substantially as shown in FIG. 1.

In yet another embodiment, the polymorphic form has a differential scanning calorimetry (DSC) curve comprising an endotherm centered from about 290° C. to about 300° C. In some variations the endotherm is centered at about 293° C. In further variations, the DSC curve is substantially as shown in FIG. 2.

### Form B:

In still another embodiment, the polymorphic form is an anhydrate having an X-ray powder diffraction pattern (CuKα) comprising significant diffraction peaks at about 5.1, 10.3 and 15.4 °2θ. In some variations, the X-ray powder diffraction pattern further comprises significant diffraction peaks at about 20.6, 24.2 and 31.8 °2θ. In further variations, the X-ray diffraction pattern is substantially as shown in FIG. 6.

### Form C:

In a still further embodiment, the polymorphic form is an anhydrate having an X-ray powder diffraction pattern (CuKα) comprising significant diffraction peaks at about 5.4, 20.2 and 20.8 °2θ. In some variations, the X-ray powder diffraction pattern further comprises significant diffraction peaks at about 4.9, 16.2 and 25.3 °2θ. In other variations, the X-ray diffraction pattern is substantially as shown in FIG. 7.

In another embodiment, the polymorphic form is an anhydrate having a differential scanning calorimetry (DSC) curve comprising an endotherm centered from about 285° C. to about 295° C. In some variations, the endotherm is centered at about 291° C. In other variations, the polymorphic form has a DSC curve substantially as shown in FIG. 8.

### Form D:

In still another embodiment, the polymorphic form is an anhydrate having an X-ray powder diffraction pattern (CuKα) comprising significant diffraction peaks at about 5.3, 6.2 and 17.4 °2θ. In some variations, the X-ray powder diffraction pattern further comprises significant diffraction peaks at about 8.9, 9.8 and 20.0 °2θ. In other variations, the X-ray diffraction pattern is substantially as shown in FIG. 12.

In a further embodiment, the polymorphic form is an anhydrate having a differential scanning calorimetry (DSC) curve comprising an endotherm centered from about 286° C. to about 296° C. In some variations, the endotherm is centered at about 282° C. In other variations, the polymorphic form has a DSC curve substantially as shown in FIG. 13.

### Form E:

In yet a further embodiment, the polymorphic form is an anhydrate having an X-ray powder diffraction pattern (CuKα) comprising significant diffraction peaks at about 5.1, 5.3 and 16.4 °2θ. In some variations, the X-ray powder diffraction pattern further comprises significant diffraction peaks at about 9.7 and 20.8 °2θ. In other variations, the X-ray diffraction pattern is substantially as shown in FIG. 16.

In another embodiment, the polymorphic form is an anhydrate having a differential scanning calorimetry (DSC) curve comprising an endotherm centered from about 287° C. to about 294° C. In some variations, the endotherm is centered at about 291° C. In some variations, a second endotherm is centered about 276° C. In other variations, the polymorphic form has a DSC curve substantially as shown in FIG. 17.

### Form F:

In still another embodiment, the polymorphic form is an anhydrate having an X-ray powder diffraction pattern (CuKα) comprising significant diffraction peaks at about 6.6, 16.7 and 17.1 °2θ. In some variations, the X-ray powder diffraction pattern further comprises significant diffraction peaks at about 6.2 and 11.2 °2θ. In other variations, the X-ray diffraction pattern is substantially as shown in FIG. 20.

In yet another embodiment, the polymorphic form is an anhydrate having a differential scanning calorimetry (DSC) curve comprising two endotherms centered from about 285° C. to about 295° C. and from about 295° C. to about 305° C., respectively. In one variation, one of the two endotherms is centered at about 289° C. and the other of the two endotherms is centered at about 299° C. In another variation, the polymorphic form has a DSC curve substantially as shown in FIG. 21.

### Form G:

In still yet another embodiment, the polymorphic form is an anhydrate having an X-ray powder diffraction pattern (CuKα) comprising significant diffraction peaks at about 15.9, 17.1 and 21.4 °2θ. In some variations, the X-ray powder diffraction pattern further comprises significant diffraction peaks at about 21.0 and 22.2 °2θ. In other variations, the X-ray diffraction pattern is substantially as shown in FIG. 23.

In a further embodiment, the polymorphic form is an anhydrate having a differential scanning calorimetry (DSC) curve comprising an endotherm centered from about 295° C. to about 305° C. In some variations, the endotherm is centered at about 299° C. In other variations, the polymorphic form has a DSC curve substantially as shown in FIG. 24.

### Salt of Compound I

In a further aspect, the invention provides a salt of Compound I of the formula:

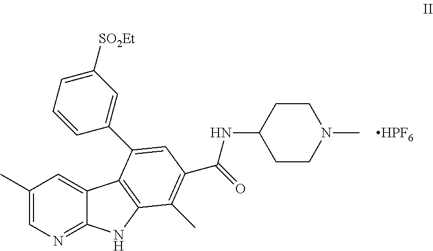

In some embodiments, the invention provides a polymorphic form of this salt, wherein the polymorphic form has an X-ray powder diffraction pattern (CuKα) comprising significant diffraction peaks at about 5.3, 10.6 and 19.6 °2θ. In one variation, the X-ray powder diffraction pattern (CuKα) further comprises significant diffraction peaks at about 10.3, 15.9 and 17.7 °2θ. In another variation, the X-ray diffraction pattern (CuKα) is substantially as shown in FIG. 26.

In other embodiments, the polymorphic form has a differential scanning calorimetry (DSC) curve comprising an endotherm centered from about 275° C. to about 285° C. In one variation, the endotherm is centered at about 281° C. In another variation, the differential scanning calorimetry (DSC) curve is substantially as shown in FIG. 27.

In another embodiment, the invention provides a method of making this salt, wherein the method comprises treating Compound I with HPF₆.

### Methods of Making Polymorphic Forms

In another aspect, the invention provides methods of making polymorphic forms of Compound I having the formula:

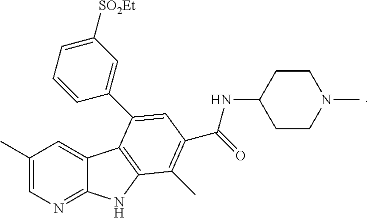

In one embodiment, the polymorphic form is Form A (e.g., a monohydrate having an X-ray powder diffraction pattern (CuKα) comprising significant diffraction peaks at about 5.4, 17.3 and 20.2 °2θ), and the method comprises treating Compound I with water. In some variations, the method further comprises dissolving Compound I in acetonitrile. In other variations, the method further comprises slurrying Compound I in acetonitrile.

In one embodiment, the polymorphic form is Form B (e.g., an anhydrate having an X-ray powder diffraction pattern (CuKα) comprising significant diffraction peaks at about 5.1, 10.3 and 15.4 °2θ), and the method comprises treating Compound I with water. In some variations, the method further comprises dissolving Compound I in acetonitrile. In other variations, the method further comprises slurrying Compound I in acetonitrile.

In a further embodiment, the polymorphic form is Form C (e.g., an anhydrate having an X-ray powder diffraction pattern (CuKα) comprising significant diffraction peaks at about 5.4, 20.2 and 20.8 °2θ), and the method comprises drying Compound I. In some variations, the method further comprises drying Compound I at a temperature above 50° C. In other variations, the method further comprises drying Compound I at a temperature above 70° C. In another variation, the method comprises treating Compound I with an atmosphere with <20% humidity. In yet another variation, the method comprises dissolving Compound I in dimethylformamide (DMF) and adding of an antisolvent to Compound I dissolved in DMF, wherein the antisolvent is selected from the group consisting of acetonitrile, ethyl acetate, isopropyl acetate, cyclohexane, heptane, and methyl tetrahydrofuran. In still yet another variation, the method comprises dissolving Compound I in an anhydrous solvent.

In still a further embodiment, the polymorphic form is Form D (e.g., an anhydrate having an X-ray powder diffraction pattern (CuKα) comprising significant diffraction peaks at about 5.3, 6.2 and 17.4 °2θ), and the method comprises treating Compound I with dioxane. In some variations of this embodiment, the method further comprises slurrying Compound I in dioxane.

In another embodiment, the polymorphic form is Form E (e.g., an anhydrate having an X-ray powder diffraction pattern (CuKα) comprising significant diffraction peaks at about 5.1, 5.3 and 16.4 °2θ), and the method comprises treating Compound I with acetone. In some variations of this embodiment, the method further comprises slurrying Compound I in acetone.

In yet another embodiment, the polymorphic form is Form F (e.g., an anhydrate having an X-ray powder diffraction pattern (CuKα) comprising significant diffraction peaks at about 6.6, 16.7 and 17.1 °2θ), and the method comprises heating Compound I. In some variations of this embodiment, the method further comprises heating Compound I at a temperature >200° C. In some variation, the method further comprises holding temperature at about 280° C.

In a further embodiment, the polymorphic form is Form G (e.g., an anhydrate having an X-ray powder diffraction pattern (CuKα) comprising significant diffraction peaks at about 15.9, 17.1 and 21.4 °2θ), and the method comprises heating Compound I. In some variations of this embodiment, the method further comprises heating Compound I at a temperature >200° C. In some variation, the method further comprises holding temperature at about 290° C.

Methods by which the above referenced analyses were performed in order to identify these physical characteristics are described in the Examples section below.

### Compositions Comprising Compound I

In a further aspect, the invention provides pharmaceutical compositions comprising

Compound I of the formula:

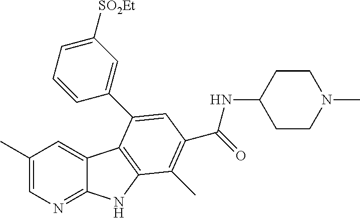

wherein at least a portion of Compound I is present as a polymorphic form, such as any polymorphic form described throughout this application.

In some embodiments, Compound I is present in a form selected from the group consisting of Form A, Form B, Form C, Form D, Form E, Form F and Form G. The present invention also relates to compositions comprising the HPF₆ salt of Compound I. It is noted that other crystalline and amorphous forms of Compound I may also be present in the composition.

In one variation, the composition comprises at least 0.1%, 0.25%, 0.5%, 1%, 5%, 10%, 25%, 50%, 75%, 80%, 85%, 90%, 95%, 97%, or 99% of Compound I where greater than 0.1%, 0.25%, 0.5%, 1%, 5%, 10%, 25%, 50%, 75%, 80%, 85%, 90%, 95%, 97% or 99% of Compound I (by weight) is present in the composition in a form selected from the group consisting of Form A, Form B, Form C, Form D, Form E, Form F and/or Form G. The composition may optionally be a pharmaceutical composition. The pharmaceutical composition may optionally further include one or more additional components that do not deleteriously affect the use of Compound I.

### Kits and Articles of Manufacture Comprising Compound I

The invention also provides kits and other articles of manufacture comprising a composition that comprises Compound I, wherein Compound I is present in a form selected from the group consisting of Form A, Form B, Form C, Form D, Form E, Form F and/or Form G. In one variation, the composition comprises at least 0.1%, 0.25%, 0.5%, 1%, 5%, 10%, 25%, 50%, 75%, 80%, 85%, 90%, 95%, 97%, or 99% of Compound I where greater than 0.1%, 0.25%, 0.5%, 1%, 5%, 10%, 25%, 50%, 75%, 80%, 85%, 90%, 95%, 97% or 99% of Compound I (by weight) is present in the composition in a form selected from the group consisting of Form A, Form B, Form C, Form D, Form E, Form F and/or Form G. The composition in the kits and articles of manufacture may optionally be a pharmaceutical composition. The pharmaceutical composition may optionally further include one or more additional components that do not deleteriously affect the use of Compound I.

In regard to each of the above embodiments including a pharmaceutical composition, the pharmaceutical composition may be formulated in any manner where at least a portion of Compound I is present in a form selected from the group consisting of Form A, Form B, Form C, Form D, Form E, Form F and/or Form G. Optionally, a portion of Compound I is present in a form selected from the group consisting of Form A, Form B, Form C, Form D, Form E, Form F and/or Form G for a period of time subsequent to administration of the pharmaceutical formulation to a subject.

Methods of Using Form A through Form G

Methods of using a pharmaceutical composition, kit and other article of manufacture comprising one or more of Form A through Form G to treat various diseases mediated by a kinase are also provided.

In one embodiment, the present invention relates to a method of inhibiting kinases comprising administering a composition where greater than 0.1%, 0.25%, 0.5%, 1%, 5%, 10%, 25%, 50%, 75%, 80%, 85%, 90%, 95%, 97% or 99% of Compound I (by weight) is present in the composition in a form selected from the group consisting of Form A, Form B, Form C, Form D, Form E, Form F and Form G. Optionally, the composition comprises at least 0.1%, 0.25%, 0.5%, 1%, 5%, 10%, 25%, 50%, 75%, 80%, 85%, 90%, 95%, 97%, or 99% of Compound I.

In another embodiment, the present invention relates to a method of inhibiting kinases in a subject (e.g., human body) with Compound I by administering Compound I where greater than 0.1%, 0.25%, 0.5%, 1%, 5%, 10%, 25%, 50%, 75%, 80%, 85%, 90%, 95%, 97% or 99% of Compound I (by weight) is present in the composition in a form selected from the group consisting of Form A, Form B, Form C, Form D, Form E, Form F and Form G, when the compound is administered. Optionally, the composition comprises at least 0.1%, 0.25%, 0.5%, 1%, 5%, 10%, 25%, 50%, 75%, 80%, 85%, 90%, 95%, 97%, or 99% of Compound I.

In another embodiment, the present invention relates to a method of inhibiting kinases in a subject (e.g., human body) with Compound I by administering Compound I where greater than 0.1%, 0.25%, 0.5%, 1%, 5%, 10%, 25%, 50%, 75%, 80%, 85%, 90%, 95%, 97% or 99% of Compound I (by weight) is present in the composition in a form selected from the group consisting of Form A, Form B, Form C, Form D, Form E, Form F and Form G for a period of time after the compound has been administered to a subject. Optionally, the composition comprises at least 0.1%, 0.25%, 0.5%, 1%, 5%, 10%, 25%, 50%, 75%, 80%, 85%, 90%, 95%, 97%, or 99% of Compound I.

In still another embodiment, the present invention provides a method of treating a disease state for which kinases possess activity that contributes to the pathology and/or symptomology of the disease state, comprising administering to a subject (e.g., human body) a composition where greater than 0.1%, 0.25%, 0.5%, 1%, 5%, 10%, 25%, 50%, 75%, 80%, 85%, 90%, 95%, 97% or 99% of Compound I (by weight) is present in the composition in a form selected from the group consisting of Form A, Form B, Form C, Form D, Form E, Form F and Form G when administered. Optionally, the composition comprises at least 0.1%, 0.25%, 0.5%, 1%, 5%, 10%, 25%, 50%, 75%, 80%, 85%, 90%, 95%, 97%, or 99% of Compound I.

In still another embodiment, the present invention provides a method of treating a disease state for which kinases possess activity that contributes to the pathology and/or symptomology of the disease state, comprising causing a composition to be present in a subject (e.g., human body) where greater than 0.1%, 0.25%, 0.5%, 1%, 5%, 10%, 25%, 50%, 75%, 80%, 85%, 90%, 95%, 97% or 99% of Compound I (by weight) is present in the composition in a form selected from the group consisting of Form A, Form B, Form C, Form D, Form E, Form F and Form G, for a period of time after the composition has been administered to a subject. Optionally, the composition comprises at least 0.1%, 0.25%, 0.5%, 1%, 5%, 10%, 25%, 50%, 75%, 80%, 85%, 90%, 95%, 97%, or 99% of Compound I.

In another embodiment, a method is provided for preventing, delaying the progression of, and/or treating conditions mediated by kinases, in particular cancer (e.g., squamous cell carcinoma, astrocytoma, Kaposi's sarcoma, glioblastoma, small-cell lung cancer, non small-cell lung cancers (e.g., large cell lung cancer, adenocarcinoma and squamous cell carcinoma), bladder cancer, head and neck cancer, melanoma, ovarian cancer, prostate cancer, breast cancer, glioma, colorectal cancer, genitourinary cancer, gastrointestinal cancer, thyroid cancer, skin cancer and blood cancers (e.g., multiple myeloma, chronic myelogenous leukemia and acute lymphocytic leukemia)); inflammation; inflammatory bowel disease; psoriasis; transplant rejection; amyotrophic lateral sclerosis; corticobasal degeneration; Down syndrome; Huntington's Disease; Parkinson's Disease; postencephelatic parkinsonism; progressive supranuclear palsy; Pick's Disease; Niemann-Pick's Disease; stroke; head trauma; chronic neurodegenerative diseases; Bipolar Disease; affective disorders; depression; schizophrenia; cognitive disorders; hair loss; contraceptive medication; mild Cognitive Impairment; Age-Associated Memory Impairment; Age-Related Cognitive Decline; Cognitive Impairment No Dementia; mild cognitive decline; mild neurocognitive decline; Late-Life Forgetfulness; memory impairment; cognitive impairment; androgenetic alopecia; dementia related diseases (e.g., Frontotemporal dementia Parkinson's Type, Parkinson dementia complex of Guam, HIV dementia, diseases with associated neurofibrillar tangle pathologies, predemented states, vascular dementia, dementia with Lewy bodies, Frontotemporal dementia and dementia pugilistica); Alzheimer's Disease; arthritis; and others.

In each instance where it is stated that Compound I may be present in the composition in a form selected from the group consisting of Form A, Form B, Form C, Form D, Form E, Form F and Form G, it is intended for the invention to encompass compositions where only one form is present, where two forms are present (all combinations) and where three, four or more forms are present (all combinations).

### BRIEF DESCRIPTION OF THE FIGURES

FIG. 1 is an XRPD pattern and peak list of Compound I Form A.

FIG. 2 is a DSC curve of Compound I Form A.

FIG. 3 is a TGA curve of Compound I Form A.

FIG. 4 ¹H NMR spectrum and peak list of Compound I Form A.

FIG. 5: Moisture sorption curve of Compound I Form A.

FIG. 6: XRPD pattern of Compound I Form B.

FIG. 7: XRPD pattern and peak list of Compound I Form C.

FIG. 8: DSC curve of Compound I Form C.

FIG. 9: TGA curve of Compound I Form C.

FIG. 10: ¹H NMR spectrum and peak list of Compound I Form C.

FIG. 11: Moisture sorption curve of Compound I Form C.

FIG. 12: XRPD pattern and peak list of Compound I Form D

FIG. 13: DSC curve of Compound I Form D.

FIG. 14: TGA curve of Compound I Form D.

FIG. 15: ¹H NMR spectrum and peak list of Compound I Form D.

FIG. 16: XRPD pattern and peak list of Compound I Form E.

FIG. 17: DSC curve of Compound I Form E.

FIG. 18: TGA curve of Compound I Form E.

FIG. 19: ¹H NMR spectrum and peak list of Compound I Form E.

FIG. 20: XRPD pattern and peak list of Compound I Form F.

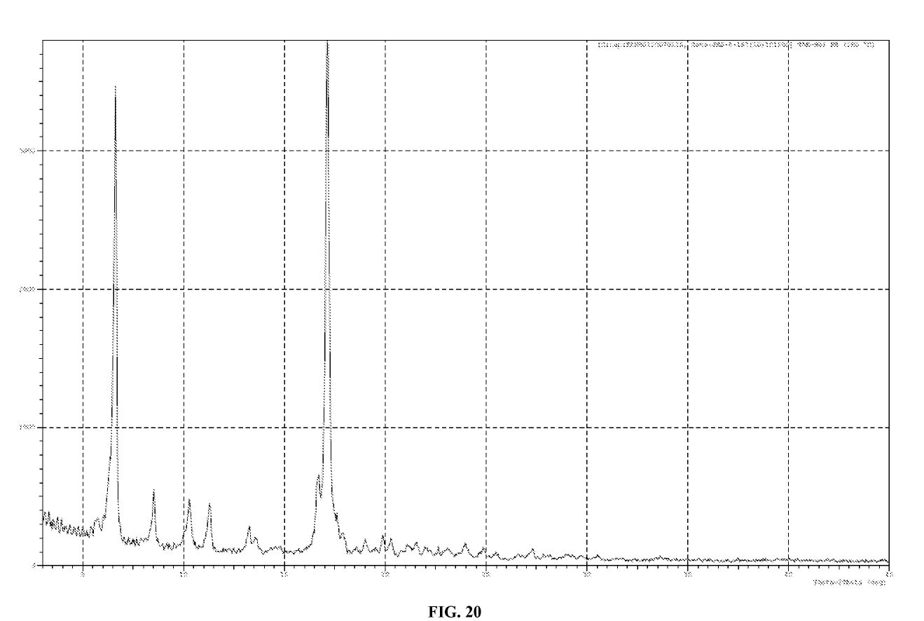

FIG. 21: DSC curve of Compound I Form F.

FIG. 22: ¹H NMR spectrum and peak list of Compound I Form F.

FIG. 23: XRPD pattern and peak list of Compound I Form G.

FIG. 24: DSC curve of Compound I Form G.

FIG. 25: ¹H NMR spectrum and peak list of Compound I Form G.

FIG. 26: XRPD pattern and peak list of Compound I HPF₆ Salt.

FIG. 27: DSC curve of Compound I HPF₆ Salt.

FIG. 28: TGA curve of Compound I HPF₆ Salt.

FIG. 29: ¹H NMR spectrum and peak list of Compound I HPF₆ Salt.

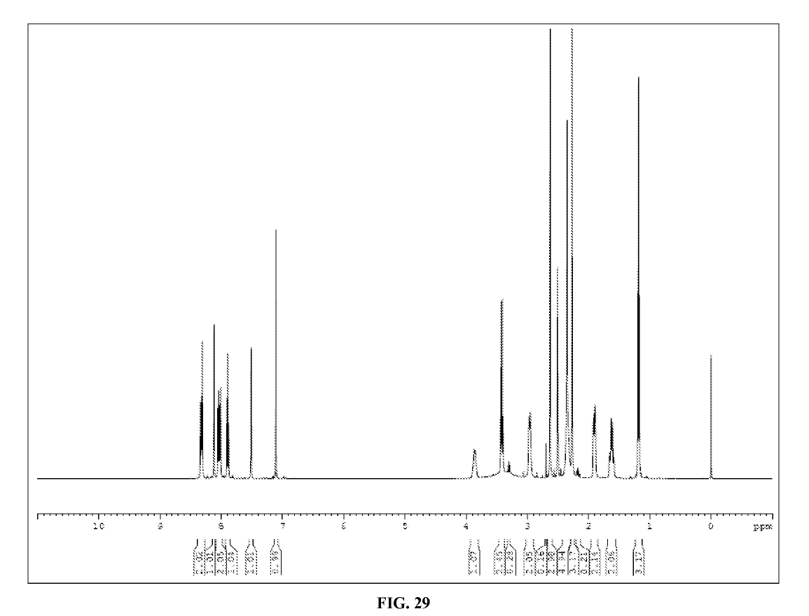

FIG. 30: ¹⁹F NMR spectrum and peak list of Compound I HPF₆ Salt.

FIG. 31: ³¹P NMR spectrum and peak list of Compound I HPF₆ Salt.

FIG. 32: Diagram of form inter-conversions relative to Form A of Compound I.

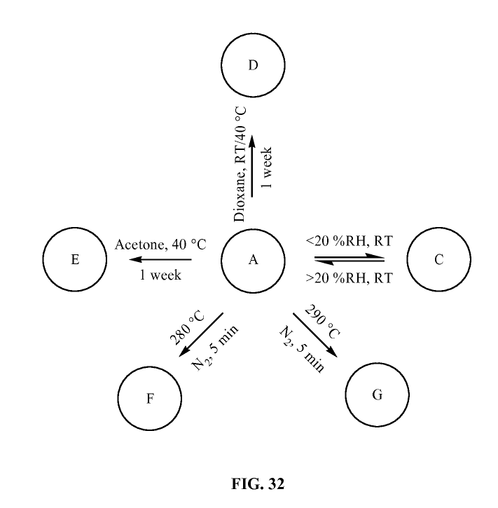

### DETAILED DESCRIPTION OF THE INVENTION

The present invention provides novel polymorphs of Compound I, as well as compositions comprising Compound I where at least a portion of Compound I is present in the composition in a form selected from the group consisting of crystalline forms (i.e., Form A, Form B, Form C, Form D, Form E, Form F and Form G) and amorphous forms.

The present invention also provides an HPF₆ salt of Compound I, as well as compositions comprising Compound I where at least a portion of Compound I is present as an HPF₆ salt.

Also provided are kits and other articles of manufacture with compositions comprising Compound I where at least a portion of Compound I is present in the composition in a form selected from the group consisting of crystalline forms (i.e., Form A, Form B, Form C, Form D, Form E, Form F and Form G) and amorphous forms.

Various methods are also provided including methods of making each of the disclosed forms; methods for manufacturing pharmaceutical compositions comprising Compound I where at least a portion of Compound I is present in the composition in a form selected from the group consisting of crystalline forms (i.e., Form A, Form B, Form C, Form D, Form E, Form F and Form G) and amorphous forms; and methods of using compositions comprising Compound I where at least a portion of Compound I is present in the composition in a form selected from the group consisting of crystalline forms (i.e., Form A, Form B, Form C, Form D, Form E, Form F and Form G) and amorphous forms.

As one will appreciate, depending on how a composition comprising a given compound is produced and then, once produced, how the composition is stored and manipulated, will influence the crystalline content of the composition. Accordingly, it is possible for a composition to comprise no crystalline content or may comprise higher concentrations of crystalline content.

It is further noted that a compound may be present in a given composition in one or more different polymorphic forms, as well as optionally also being present as an amorphous material. This may be the result of (a) physically mixing two or more different polymorphic forms; (b) having two or more different polymorphic forms be generated from crystallization conditions; (c) having all or a portion of a given polymorphic form convert into another polymorphic form; (d) having all or a portion of a compound in an amorphous state convert into two or more polymorphic forms; as well as for a host of other reasons.

As can be seen, depending on how a composition comprising a compound is prepared, the percentage, by weight, of that compound in a given polymorphic form can vary from 0% to 100%. According to the present invention, compositions are provided where greater than 0.1%, 0.25%, 0.5%, 1%, 5%, 10%, 25%, 50%, 75%, 80%, 85%, 90%, 95%, 97%, or 99% or more of Compound I (by weight) is present in the composition in a form selected from the group consisting of Forms A to G.

### Definitions

“Crystalline”, as the term is used herein, refers to a material that contains a specific compound, which may be hydrated and/or solvated, and has sufficient crystalline content to exhibit a discernable diffraction pattern by XRPD or other diffraction techniques. Often, a crystalline material that is obtained by direct crystallization of a compound dissolved in a solution or interconversion of crystals obtained under different crystallization conditions, will have crystals that contain the solvent used in the crystallization, termed a crystalline solvate. Also, the specific solvent system and physical embodiment in which the crystallization is performed, collectively termed crystallization conditions, may result in the crystalline material having physical and chemical properties that are unique to the crystallization conditions, generally due to the orientation of the chemical moieties of the compound with respect to each other within the crystal and/or the predominance of a specific polymorphic form of the compound in the crystalline material.

Depending upon the polymorphic form(s) of the compound that are present in a composition, various amounts of the compound in an amorphous solid state may also be present, either as a side product of the initial crystallization, and/or a product of degradation of the crystals comprising the crystalline material. Thus, crystalline, as the term is used herein, contemplates that the composition may include amorphous content; the presence of the crystalline material among the amorphous material being detectable by, among other methods, the composition having a diffraction pattern with individual, discernable peaks.

The amorphous content of a crystalline material may be increased by grinding or pulverizing the material, which is evidenced by broadening of diffraction and other spectral lines relative to the crystalline material prior to grinding. Sufficient grinding and/or pulverizing may broaden the lines relative to the crystalline material prior to grinding to the extent that the XRPD or other crystal specific spectrum may become indiscernible, making the material substantially amorphous or quasi-amorphous.

Continued grinding would be expected to increase the amorphous content and further broaden the XRPD pattern with the limit of the XRPD pattern being so broadened that it can no longer be discerned above noise. When the XRPD pattern is broadened to the limit of being indiscernible, the material may be considered to no longer be a crystalline material, but instead be wholly amorphous. For material having increased amorphous content and wholly amorphous material, no peaks should be observed that would indicate grinding produces another form.

“Amorphous”, as the term is used herein, refers to a composition comprising a compound that contains too little crystalline content of the compound to yield a diffraction pattern, by XRPD or other diffraction techniques, having individual, discernable peaks. Glassy materials are a type of amorphous material. Glassy materials do not have a true crystal lattice, and technically resemble very viscous non-crystalline liquids. Rather than being true solids, glasses may better be described as quasi-solid amorphous material.

“Broad” or “broadened”, as the term is used herein to describe spectral lines, including XRPD, NMR, IR and Raman spectroscopy lines, is a relative term that relates to the line width of a baseline spectrum. The baseline spectrum is often that of an unmanipulated crystalline form of a specific compound as obtained directly from a given set of physical and chemical conditions, including solvent composition and properties such as temperature and pressure. For example, broadened can be used to describe the spectral lines of a XRPD spectrum of ground or pulverized material comprising a crystalline compound relative to the material prior to grinding. In materials where the constituent molecules, ions or atoms, as solvated or hydrated, are not tumbling rapidly, line broadening is indicative of increased randomness in the orientation of the chemical moieties of the compound, thus indicative of an increased amorphous content. When comparisons are made between crystalline materials obtained via different crystallization conditions, broader spectral lines indicate that the material producing the relatively broader spectral lines has a higher level of amorphous material.

“About” as the term is used herein, refers to an estimate that the actual value falls within ±5% of the value cited.

“Forked” as the term is used herein to describe DSC endotherms and exotherms, refers to overlapping endotherms or exotherms having distinguishable peak positions.

### Preparation and Characterization of the Polymorphs

A. Preparation of Compound I

Various methods may be used to synthesize Compound I. A representative method for synthesizing Compound I is provided in Example 1. It is noted, however, that other synthetic routes may also be used to synthesize Compound I.

B. Preparation of the Polymorphs of Compound I

General methods for precipitating and crystallizing a compound may be applied to prepare the various polymorphs described herein. These general methods are known to those skilled in the art of synthetic organic chemistry and pharmaceutical formulation, and are described, for example, by J. March, “Advanced Organic Chemistry: Reactions, Mechanisms and Structure,” 4^{th} Ed. (New York: Wiley-Interscience, 1992).

In general, a given polymorph of a compound may be obtained by direct crystallization of the compound or by crystallization of the compound followed by inter-conversion from another polymorphic form or from an amorphous form. Depending on the method by which a compound is crystallized, the resulting composition may contain different amounts of the compound in crystalline form as opposed to as an amorphous material. Also, the resulting composition may contain differing mixtures of different polymorphic forms of the compound.

Compositions comprising a higher percentage of crystalline content (e.g., forming crystals having fewer lattice defects and proportionately less glassy material) are generally prepared when conditions are used that favor slower crystal formation, including those slowing solvent evaporation and those affecting kinetics. Crystallization conditions may be appropriately adjusted to obtain higher quality crystalline material as necessary. Thus, for example, if poor crystals are formed under an initial set of crystallization conditions, the solvent temperature may be reduced and ambient pressure above the solution may be increased relative to the initial set of crystallization conditions in order to slow crystallization.

Precipitation of a compound from solution, often affected by rapid evaporation of solvent, is known to favor the compound forming an amorphous solid as opposed to crystals. A compound in an amorphous state may be produced by rapidly evaporating solvent from a solvated compound, or by grinding, pulverizing or otherwise physically pressurizing or abrading the compound while in a crystalline state.

Compound I as prepared by the method described in Example 1 may be used as the starting material for preparation of other polymorphic forms. The methods for testing the solubility of Compound I are described in Example 2, and the solubilities of Compound I in various solvents are summarized in Table A of Example 2. Good solubility was observed in MeOH, DMF, DMA, NMP, CHCl₃, AcOH and EtOH. Poor solubility was observed in MeCN, MTBe, EtOAc, IPAc, IPA, THF, MEK, heptane and water.

Methods by which the various polymorphic forms may be prepared are described in Subsection A, Example 3. Specific methods by which Forms A-G and the HPF₆ salt of Compound I may be prepared are described below and in the Examples section.

C. Polymorphs of Compound I

Described herein are Form A, Form B, Form C, Form D, Form E, Form F and Form G of Compound I. Various tests were performed in order to physically characterize the polymorphs of Compound I, including X-ray powder diffraction (XRPD), differential scanning calorimetry (DSC), thermogravimetric analysis (TGA), solution proton nuclear magnetic resonance (¹H-NMR), and moisture sorption and desorption analysis (M S/Des). Detailed experimental conditions for each of the analytical techniques are described in the Examples section. Solubility test were also conducted, and Compound I displayed appreciable solubility in polar solvents including MeCN, EtOH, THF, DMF, AcOH, MeOH, NMP, and DMAc (Table 11). No solids were observed to precipitate out in both the fast and slow cooling procedures, and isolation of solid materials was accomplished through evaporation. A summary of these experiments and the results are presented below and in Table 12.

1. Form A

Based on the available characterization data, Form A appears to be a dihydrate polymorphic form of Compound I that is stable at ambient conditions.

Form A was characterized by a variety of techniques, including XRPD, DSC, TGA, ¹H-NMR, Karl Fisher, and moisture sorption analysis. Table 9 summarizes some of these results.

A detailed method for preparing Form A is presented in Example 8. For example, Form A can be obtained from a water re-slurry (e.g., a binary solvent system, such as MeCN/water).

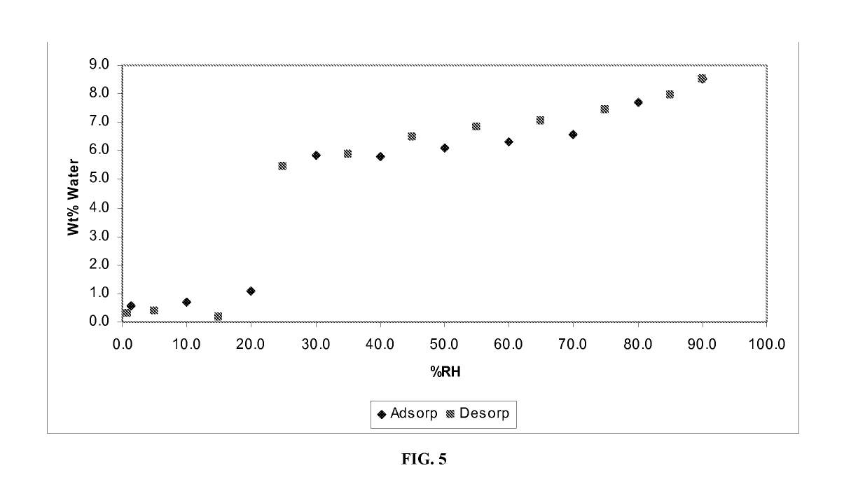

Karl Fisher analysis of Form A generally showed 5 to 6 wt % water. This data was consistent with the moisture sorption study (FIG. 5) which is itself consistent with Form A of Compound I being a dehydrate (theoretical wt % water is 6.2%).

The gravimetric moisture sorption experiment of Form A of Compound I showed the material to be a stable dihydrate above 20% RH during both adsorption and desorption. The experiment did not reach the 4-hour equilibration limit at any point. Further, the material gained enough water to form the dihydrate in approximately 45 minutes at 30% RH during adsorption and lost enough water to form the anhydrate at 15% RH during desorption in less than 60 minutes. These results are consistent with the material freely loses/gains water between 20 and 25% RH to form the anhydrate or dihydrate respectively. XRPD analysis of the solid following the desorption scan showed the material to be consistent with Form C of Compound I, consistent with a form conversion. The sample was further allowed to equilibrate at ambient laboratory conditions overnight and reanalyzed by XRPD. The results were consistent with the material having re-converted to Form A. Other polymorphic forms of Compound I can convert to Form A at about a humidity above about 20% RH.

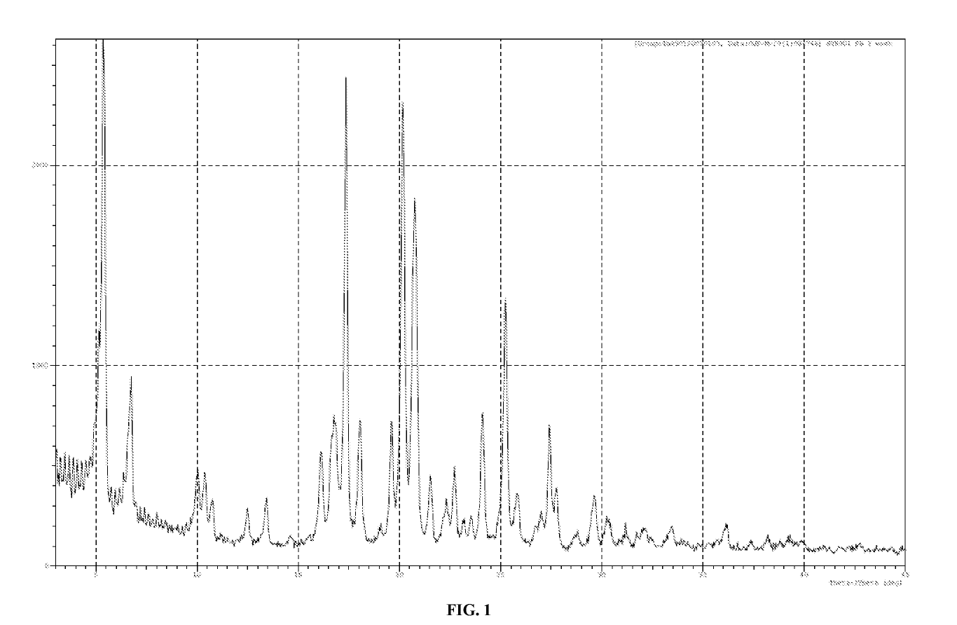

A humidity chamber study of Form A of Compound I is summarized in Table 20. A sample of Form A was exposed to ambient temperature and 0% RH for one week. XRPD analysis of the material obtained after one week showed a pattern consistent with Form A and Karl Fischer (KF) analysis results before and after the one week equilibration were comparable. These results were consistent with conversion from Form A to Form C in the 0% RH chamber and reconversion to Form A at ambient laboratory conditions during sample preparation for analysis. A summary of form inter-conversion in relation to Form A is shown in FIG. 1.

FIG. 1 shows a characteristic XRPD spectrum (CuKα, λ=1.5418 Å) of Form A. The XRPD pattern confirms that Form A is crystalline. Major X-Ray diffraction lines expressed in °2θ and their relative intensities are summarized in Table 1.

TABLE 1
Characteristic XRPD Peaks (CuKα) of Form A

| Peak No. | 2θ (°) | d-spacing | Intensity | I/Iₒ |
|---|---|---|---|---|
| 1 | 4.5200 | 19.53380 | 93 | 624 |
| 2 | 4.9200 | 17.94655 | 256 | 2505 |
| 3 | 5.3703 | 16.44269 | 1780 | 11723 |
| 4 | 6.6866 | 13.20851 | 503 | 3845 |
| 5 | 10.0068 | 8.83221 | 256 | 2132 |
| 6 | 10.3521 | 8.53837 | 250 | 1634 |
| 7 | 10.7150 | 8.24999 | 154 | 938 |
| 8 | 12.4695 | 7.09285 | 124 | 797 |
| 9 | 13.4022 | 6.60126 | 171 | 1039 |
| 10 | 16.1144 | 5.49580 | 346 | 2263 |
| 11 | 16.7419 | 5.29120 | 481 | 5667 |
| 12 | 17.3410 | 5.10972 | 1652 | 9249 |
| 13 | 18.0305 | 4.91585 | 489 | 3027 |
| 14 | 19.5947 | 4.52681 | 460 | 3154 |
| 15 | 20.1550 | 4.40221 | 1723 | 10858 |
| 16 | 20.7258 | 4.28225 | 1346 | 11226 |
| 17 | 21.5105 | 4.12777 | 245 | 1511 |
| 18 | 22.2737 | 3.98803 | 142 | 1161 |
| 19 | 22.6921 | 3.91543 | 262 | 1475 |
| 20 | 23.1463 | 3.83962 | 77 | 353 |
| 21 | 23.5235 | 3.77890 | 91 | 413 |
| 22 | 24.0830 | 3.69236 | 504 | 2821 |
| 23 | 24.8400 | 3.58152 | 77 | 385 |
| 24 | 25.2071 | 3.53019 | 955 | 5719 |
| 25 | 25.7751 | 3.45367 | 196 | 1695 |
| 26 | 26.7200 | 3.33364 | 74 | 369 |
| 27 | 26.9735 | 3.30288 | 122 | 878 |
| 28 | 27.3948 | 3.25304 | 489 | 2499 |
| 29 | 27.7055 | 3.21726 | 243 | 1591 |
| 30 | 28.7008 | 3.10791 | 58 | 424 |
| 31 | 29.5754 | 3.01797 | 202 | 1561 |
| 32 | 30.2703 | 2.95025 | 108 | 1017 |
| 33 | 31.1548 | 2.86848 | 74 | 441 |
| 34 | 32.0500 | 2.79037 | 73 | 1015 |
| 35 | 33.3745 | 2.68259 | 82 | 973 |
| 36 | 36.0772 | 2.48759 | 96 | 1047 |

This unique set of XRPD peak positions or a subset thereof can be used to identify Form A. One such subset comprises peaks at about 5.4, 17.3 and 20.2 °2θ. Another subset comprises peaks comprises peaks at about 16.7, 20.7 and 25.2 °2θ.

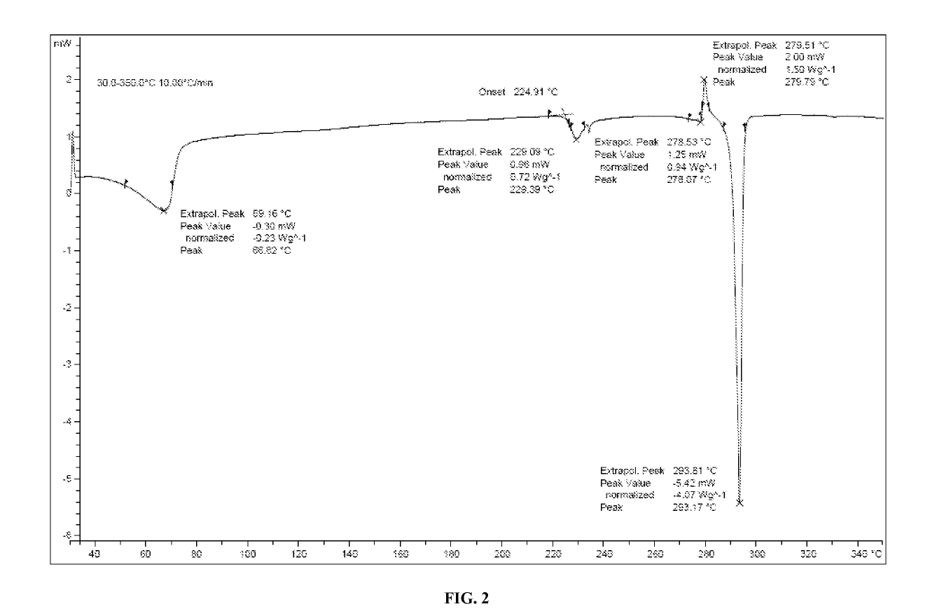

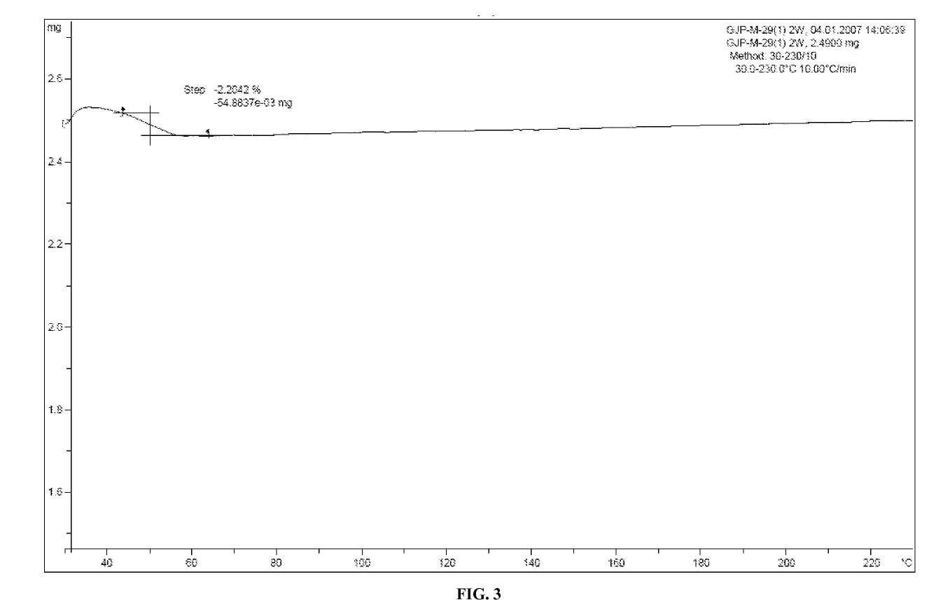

The DSC thermogram (FIG. 2) showed several endothermic events below 300° C., typically occurring near 67, 229 and 293° C. with an exothermic event sometimes observed near 280° C. Analysis for Form A by TGA (FIG. 3) typically showed a weight loss below 60° C. of 2 to 4%. Accurate determination of this weight loss was difficult due to the low onset temperature of the event.

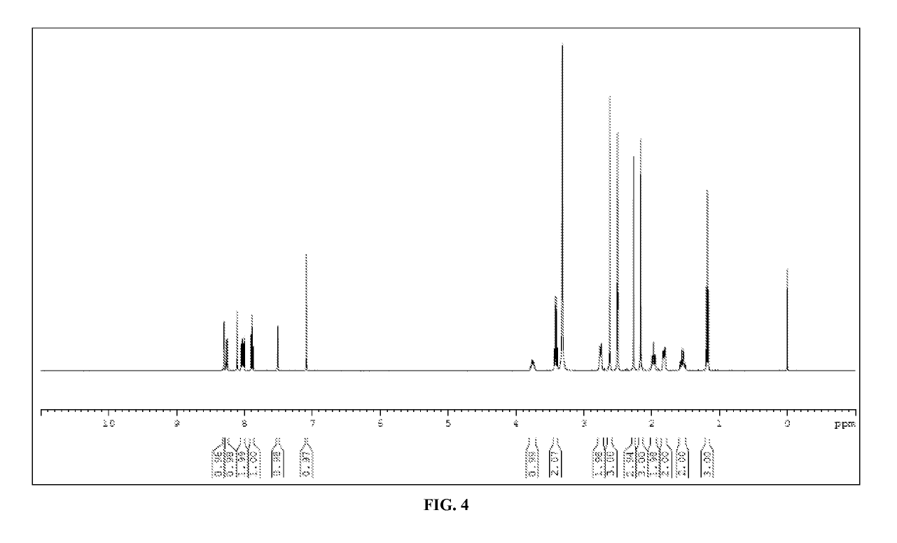

Form A was further characterized by solution ¹H NMR. The spectrum is reported in FIG. 4. Chemical assignments were not performed; however, the spectra are consistent with the known chemical structure of Compound I.

The solubility of Compound I was found to be <0.1 mg/mL in de-ionized water and in 20 mM phosphate buffer at pH 3.2 (Table 10).

Further details related to the preparation and characterization of Form A are presented below in the Examples section.

2. Form B

Based on the available characterization data, Form B appears to be an anhydrous polymeric form of Compound I that is stable under ambient non-aqueous conditions. Form B was characterized by a variety of techniques, including XRPD. Table 9 summarizes some of these results.

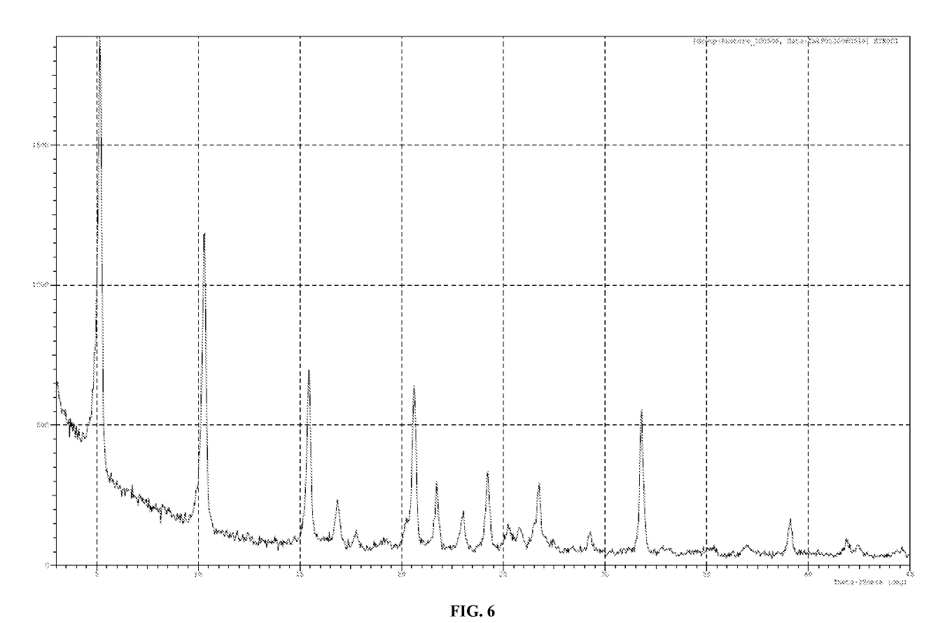

FIG. 6 shows a characteristic XRPD spectrum (CuKα, λ=1.5418 Å) of Form B. The XRPD pattern confirms that Form B is crystalline. Major X-Ray diffraction lines expressed in °2θ and their relative intensities are summarized in Table 2.

TABLE 2
Characteristic XRPD Peaks (CuKα) of Form B

| Peak No. | 2θ (°) | d-spacing | Intensity | I/Iₒ |
|---|---|---|---|---|
| 1 | 4.6000 | 19.19426 | 71 | 465 |
| 2 | 4.8400 | 18.24300 | 225 | 1211 |
| 3 | 5.1500 | 17.14555 | 1162 | 7459 |
| 4 | 9.8400 | 8.98155 | 77 | 806 |
| 5 | 10.2738 | 8.60327 | 819 | 5717 |
| 6 | 15.1200 | 5.85493 | 53 | 289 |
| 7 | 15.4203 | 5.74158 | 460 | 3071 |
| 8 | 16.8292 | 5.26395 | 118 | 1005 |
| 9 | 17.7451 | 4.99426 | 42 | 297 |
| 10 | 20.1200 | 4.40979 | 58 | 509 |
| 11 | 20.6008 | 4.30795 | 459 | 3065 |
| 12 | 21.7072 | 4.09081 | 162 | 1132 |
| 13 | 22.9794 | 3.86713 | 108 | 853 |
| 14 | 24.2136 | 3.67274 | 210 | 1543 |
| 15 | 25.2343 | 3.52644 | 66 | 620 |
| 16 | 25.7957 | 3.45096 | 61 | 500 |
| 17 | 26.4800 | 3.36331 | 74 | 431 |
| 18 | 26.7173 | 3.33397 | 192 | 1298 |
| 19 | 29.2269 | 3.05315 | 62 | 462 |
| 20 | 31.7669 | 2.81459 | 433 | 2538 |
| 21 | 39.0677 | 2.30378 | 102 | 699 |
| 22 | 41.8620 | 2.15623 | 49 | 338 |

This unique set of XRPD peak positions or a subset thereof can be used to identify Form B. One such subset comprises peaks at about 5.1, 10.3 and 15.4 °2θ. Another subset comprises peaks at about 20.6, 24.2 and 31.8 °2θ.

Further details related to the preparation and characterization of Form B are presented below in the Examples section.

3. Form C

Based on the available characterization data, Form C appears to be an anhydrous polymeric form of Compound I that is stable under ambient low humidity conditions. Form C was characterized by a variety of techniques, including XRPD, DSC, TGA, Karl Fischer, ¹H-NMR and moisture sorption analysis. Table 9 summarizes some of these results.

In general, Form C of Compound I was observed to be an anhydrate below 25% relative humidity which rapidly converted to the dihydrate Form A above 20% relative humidity. In addition, Form C converted to Form A in aqueous media during the solubility determination.

Form C can be prepared by binary solvent crystallizations using DMF as the primary solvent and a range of different anti-solvents, including MTBE, EtOAc, IPAc, 2-Me-THF, c-hexane, heptane, toluene, and water (Table 13). Form C can also be prepared by freebasing the HCl salt derivative of Compound I (Example 6). This can be accomplished by dissolving the HCl salt of Compound I and the treating the solution with base. This results in precipitation of the free base. A detailed method for preparing Form C is presented in Example 8.

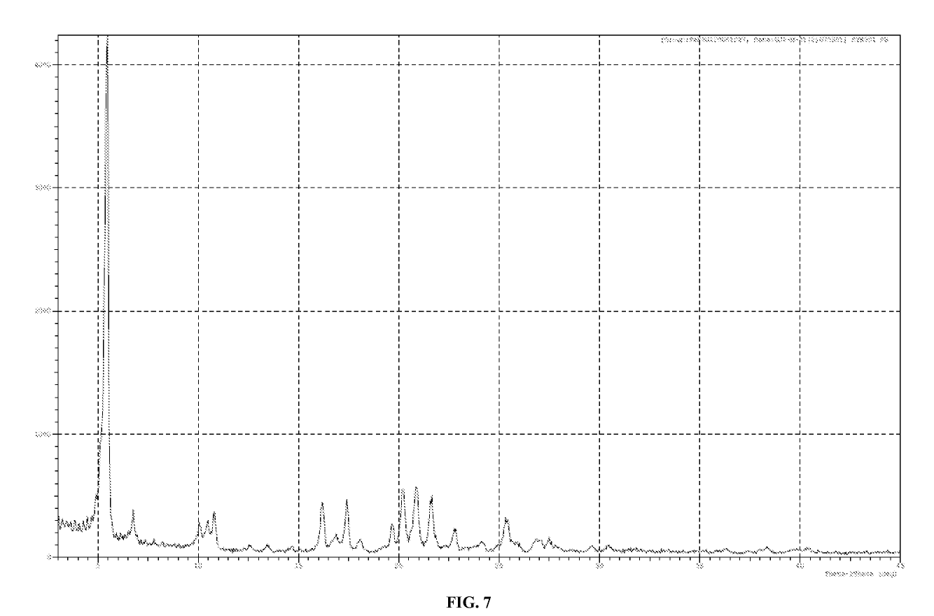

FIG. 7 shows a characteristic XRPD spectrum (CuKα, λ=1.5418 Å) of Form C. The XRPD pattern confirms that Form C is crystalline. Major X-Ray diffraction lines expressed in °2θ and their relative intensities are summarized in Table 3.

TABLE 3
Characteristic XRPD Peaks (CuKα) of Form C

| Peak No. | 2θ (°) | d-spacing | Intensity | I/Iₒ |
|---|---|---|---|---|
| 1 | 4.8800 | 18.09356 | 194 | 2586 |
| 2 | 5.4244 | 16.27882 | 3165 | 20354 |
| 3 | 6.7517 | 13.08129 | 158 | 1029 |
| 4 | 10.0680 | 8.77866 | 151 | 1249 |
| 5 | 10.4400 | 8.46668 | 165 | 1153 |
| 6 | 10.7749 | 8.20426 | 227 | 1373 |
| 7 | 16.1684 | 5.47756 | 299 | 2372 |
| 8 | 17.3885 | 5.09587 | 298 | 2223 |
| 9 | 19.6531 | 4.51349 | 162 | 1132 |
| 10 | 20.1850 | 4.39574 | 399 | 2684 |
| 11 | 20.8418 | 4.25867 | 397 | 3165 |
| 12 | 21.5879 | 4.11315 | 323 | 2270 |
| 13 | 22.7605 | 3.90382 | 127 | 849 |
| 14 | 25.3418 | 3.51173 | 202 | 2438 |

This unique set of XRPD peak positions or a subset thereof can be used to identify Form C. One such subset comprises peaks at about 5.4, 20.2 and 20.8 °2θ. Another subset comprises peaks at about 4.9, 16.2 and 25.3 °2θ.

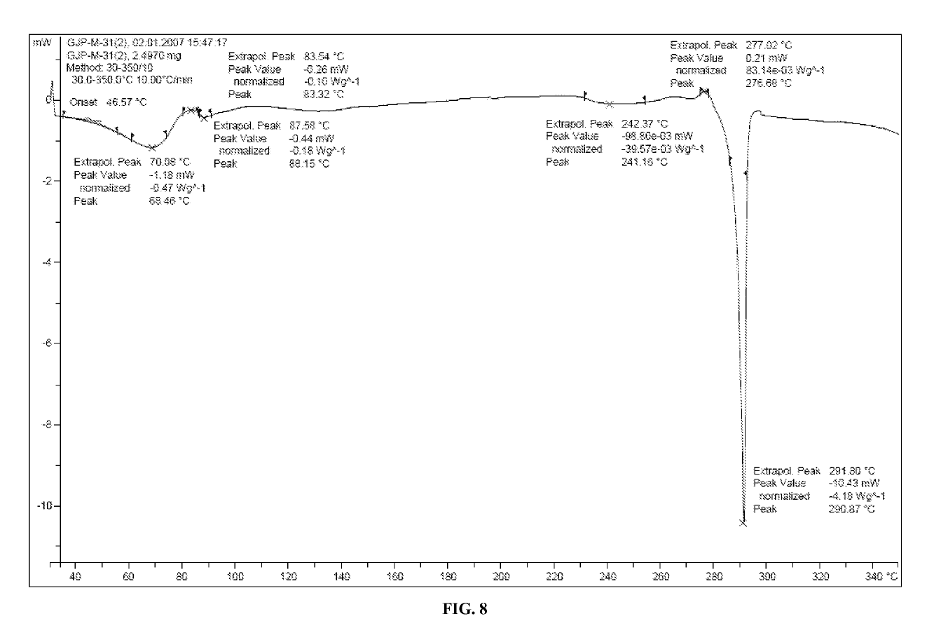

The DSC thermogram (FIG. 8) shows endothermic events near about 68 and about 291° C.

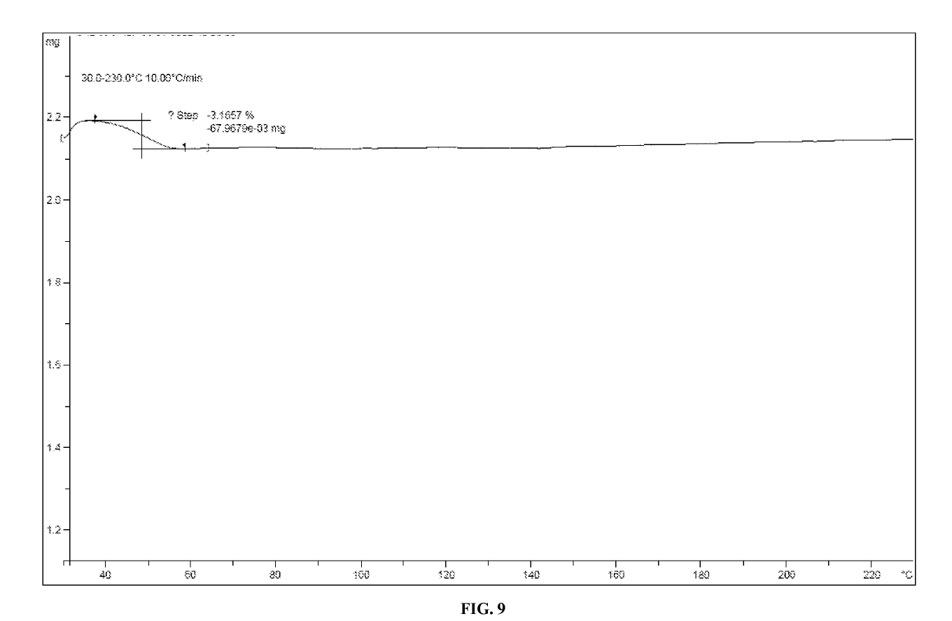

Analysis by TGA (FIG. 9) showed a variable weight loss between 0 and approximately 4 wt %. Karl Fisher analysis also showed variable water content between <1 to approximately 4%. These results may be indicative of adsorbed water or the material converted (or began to convert) to Form A which would be consistent with moisture sorption results discussed below which showed conversion to Form A between 20 and 30% RH.

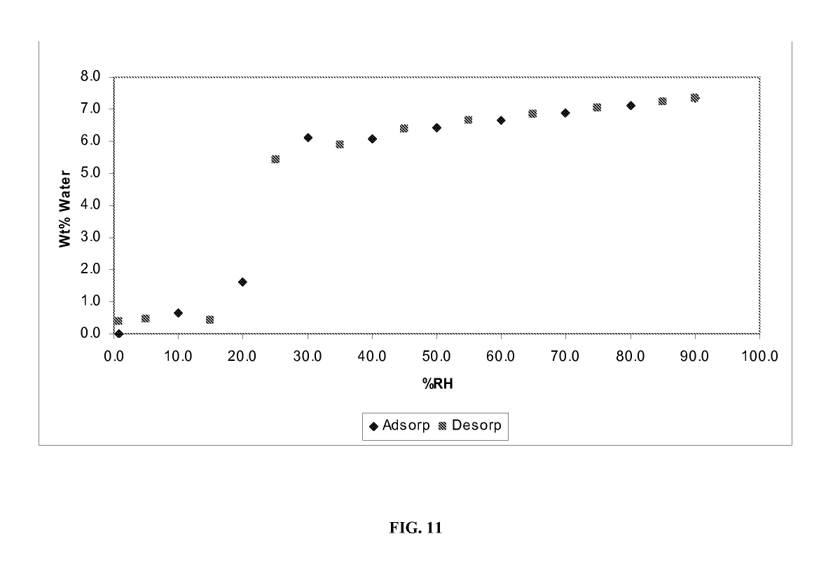

The moisture sorption experiment of Form C of Compound I provides a curve similar to the Form A curve (FIG. 11). The experiment showed the material to convert to a stable dihydrate above 20% RH during both adsorption and desorption. The experiment did not reach the 4-hour equilibration limit at any point. Further, the material gained enough water to form the dihydrate in less than 60 minutes at 30% RH during adsorption and lost enough water to form the anhydrate at 15% RH during desorption in approximately 80 minutes. These results are consistent with the material freely losing/gaining water between 20 and 25% RH to form the anhydrate or dihydrate respectively.

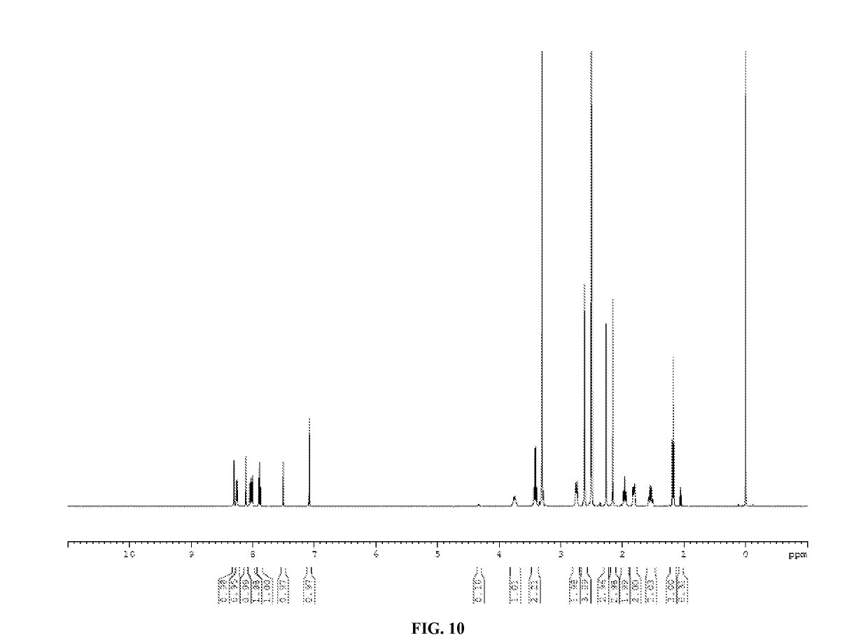

Form C was further characterized by solution ¹H NMR. The spectrum is reported in FIG. 10. Chemical assignments were not performed; however, the spectra are consistent with the known chemical structure of Compound I.

Further details related to the preparation and characterization of Form C are presented below in the Examples section.

4. Form D

Based on the available characterization data, Form D appears to be an anhydrous polymorphic form of Compound I that is stable under ambient non-aqueous conditions. Form D was characterized by techniques including XRPD, DSC, TGA, and ¹H-NMR. Table 9 summarizes some of these results. Form D can be isolated in slurry experiments in dioxane at ambient temperature and at 40° C. A detailed method for preparing Form D is presented in Example 8.

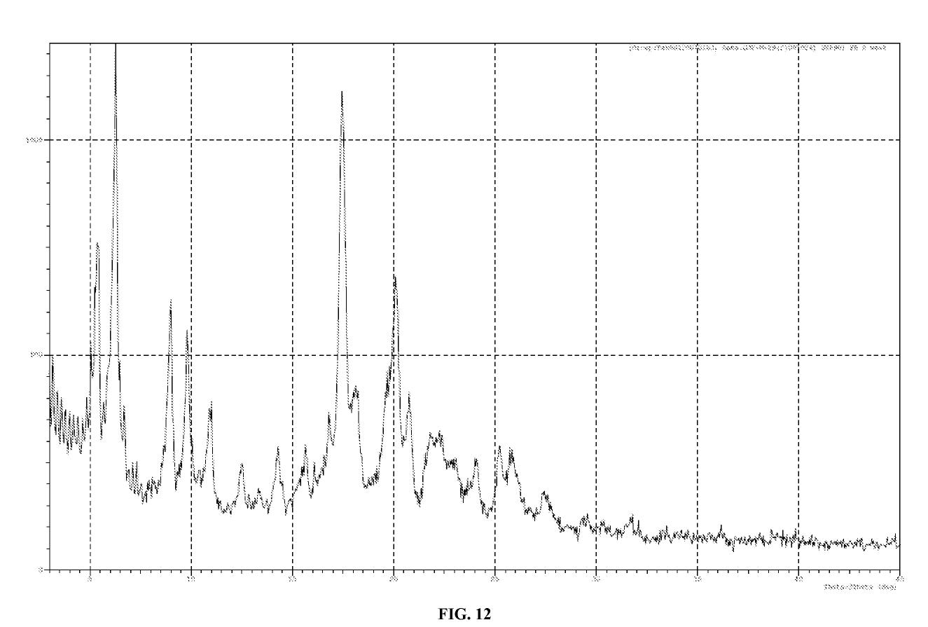

FIG. 12 shows a characteristic XRPD spectrum (CuKα, λ=1.5418 Å) of Form D. The XRPD pattern confirms that Form D is crystalline. Major X-Ray diffraction lines expressed in °2θ and their relative intensities are summarized in Table 4.

TABLE 4
Characteristic XRPD Peaks (CuKα) of Form D

| Peak No. | 2θ (°) | d-spacing | Intensity | I/Iₒ |
|---|---|---|---|---|
| 1 | 4.8800 | 18.09356 | 51 | 883 |
| 2 | 5.3388 | 16.53964 | 401 | 3352 |
| 3 | 6.2351 | 14.16392 | 680 | 6170 |
| 4 | 6.6800 | 13.22154 | 93 | 794 |
| 5 | 8.6000 | 10.27358 | 62 | 442 |
| 6 | 8.9434 | 9.87986 | 313 | 2523 |
| 7 | 9.8040 | 9.01445 | 275 | 2802 |
| 8 | 10.4000 | 8.49915 | 47 | 0 |
| 9 | 10.9036 | 8.10771 | 174 | 1703 |
| 10 | 12.4745 | 7.09002 | 77 | 606 |
| 11 | 13.3389 | 6.63245 | 27 | 181 |
| 12 | 14.2655 | 6.20366 | 93 | 804 |
| 13 | 15.2400 | 5.80910 | 27 | 191 |
| 14 | 15.6046 | 5.67418 | 81 | 585 |
| 15 | 16.1035 | 5.49949 | 38 | 294 |
| 16 | 16.8000 | 5.27303 | 123 | 1301 |
| 17 | 17.4491 | 5.07831 | 722 | 7157 |
| 18 | 18.1200 | 4.89177 | 184 | 2474 |
| 19 | 19.5200 | 4.54397 | 135 | 1216 |
| 20 | 19.9929 | 4.43754 | 381 | 4354 |
| 21 | 20.6813 | 4.29136 | 160 | 1672 |
| 22 | 21.8000 | 4.07360 | 105 | 1877 |
| 23 | 22.2800 | 3.98692 | 109 | 0 |
| 24 | 23.0000 | 3.86371 | 71 | 1785 |
| 25 | 23.6800 | 3.75428 | 36 | 293 |
| 26 | 24.0277 | 3.70073 | 93 | 750 |
| 27 | 25.2000 | 3.53117 | 124 | 949 |
| 28 | 25.7600 | 3.45566 | 111 | 2091 |
| 29 | 27.4838 | 3.24271 | 54 | 790 |
| 30 | 29.2800 | 3.04774 | 23 | 83 |
| 31 | 29.5200 | 3.02350 | 24 | 291 |
| 32 | 31.6733 | 2.82269 | 33 | 387 |

This unique set of XRPD peak positions or a subset thereof can be used to identify Form D. One such subset comprises peaks at about 5.3, 6.2 and 17.4 °2θ. Another subset comprises peaks at about 8.9, 9.8 and 20.0 °2θ.

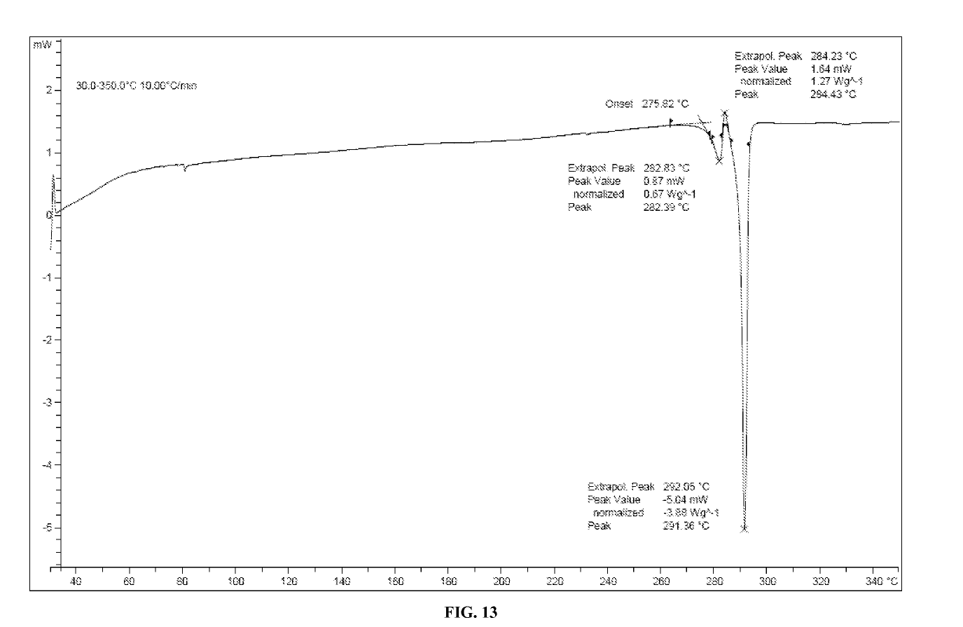

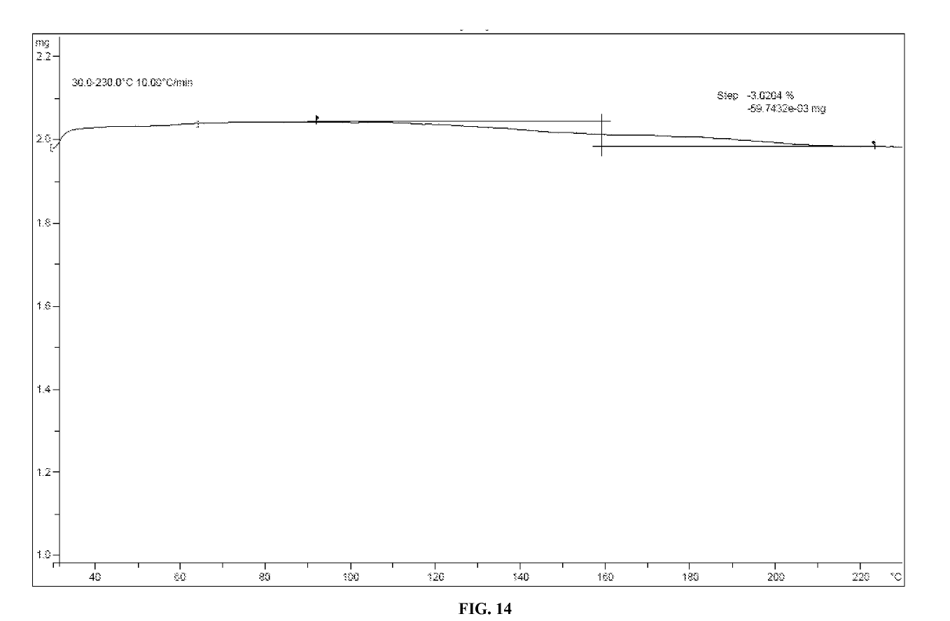

KF analysis results of a sample of Form D of Compound I found 1.4% water. A DSC thermogram (FIG. 13) showed multiple events, including endotherms at 282 and 292° C. and an exothermic event at 284° C. A TGA of a sample of Form D of Compound I (FIG. 14) showed 0.9% weight loss between 100 and 180° C.

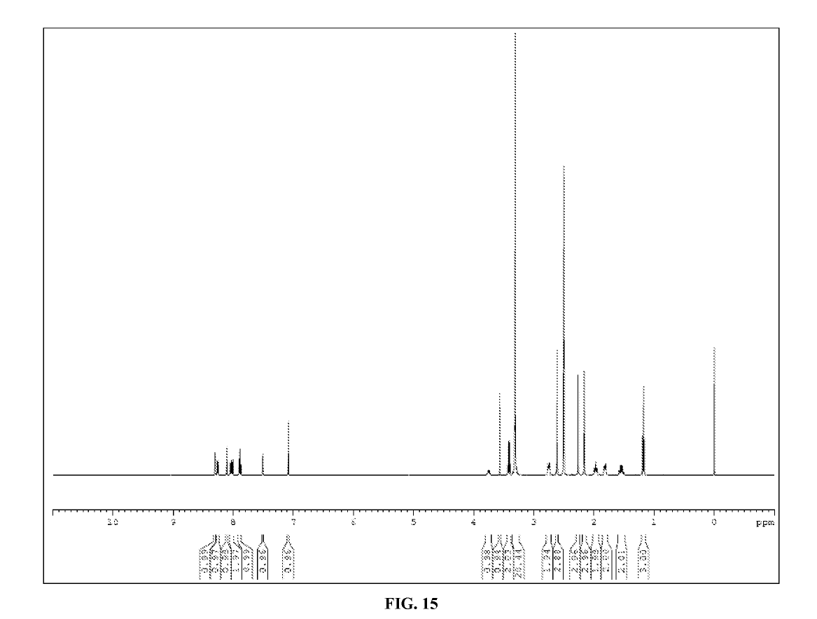

Form D was further characterized by solution ¹H NMR. The spectrum is reported in FIG. 15. Chemical assignments were not performed; however, the spectra are consistent with the known chemical structure of Compound I. The spectrum also showed residual dioxane present (1.9 wt %).

Further details related to the preparation and characterization of Form D are presented below in the Examples section.

5. Form E

Based on the available characterization data, Form E appears to be an anhydrous polymorphic form of Compound I that is stable under ambient non-aqueous conditions. Form E was characterized by techniques including XRPD, DSC, TGA, and ¹H-NMR. Table 9 summarizes some of these results. A detailed method for preparing Form E is presented in Example 8.

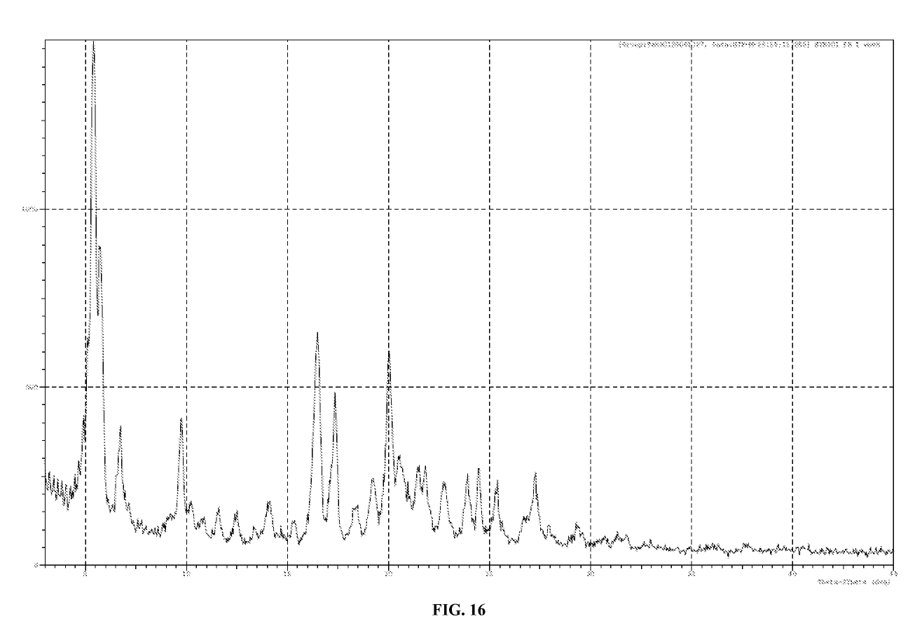

FIG. 16 shows a characteristic XRPD spectrum (CuKα, λ=1.5418 Å) of Form E. The XRPD pattern confirms that Form E is crystalline. Major X-Ray diffraction lines expressed in °2θ and their relative intensities are summarized in Table 5.

TABLE 5
Characteristic XRPD Peaks (CuKα) of Form E

| Peak No. | 2θ (°) | d-spacing | Intensity | I/Iₒ |
|---|---|---|---|---|
| 1 | 3.6695 | 24.05913 | 63 | 318 |
| 2 | 3.8808 | 22.74963 | 81 | 224 |
| 3 | 4.9200 | 17.94655 | 360 | 2822 |
| 4 | 5.1200 | 17.24595 | 682 | 3422 |
| 5 | 5.3419 | 16.53005 | 1963 | 9141 |
| 6 | 5.7600 | 15.33109 | 549 | 4246 |
| 7 | 6.5600 | 13.46313 | 89 | 402 |
| 8 | 6.7600 | 13.06525 | 76 | 417 |
| 9 | 9.0800 | 9.73153 | 139 | 1300 |
| 10 | 9.6798 | 9.12983 | 481 | 3467 |
| 11 | 10.6405 | 8.30758 | 115 | 899 |
| 12 | 11.5403 | 7.66178 | 114 | 677 |
| 13 | 14.0138 | 6.31451 | 146 | 1671 |
| 14 | 15.2349 | 5.81103 | 82 | 617 |
| 15 | 16.0400 | 5.52112 | 193 | 1178 |
| 16 | 16.3684 | 5.41108 | 963 | 7159 |
| 17 | 17.0400 | 5.19930 | 193 | 1572 |
| 18 | 17.3200 | 5.11587 | 306 | 1755 |
| 19 | 18.3065 | 4.84235 | 128 | 1017 |
| 20 | 19.2400 | 4.60946 | 102 | 781 |
| 21 | 19.6800 | 4.50738 | 204 | 1187 |
| 22 | 19.9432 | 4.44849 | 622 | 4506 |
| 23 | 20.5364 | 4.32131 | 277 | 2751 |
| 24 | 20.8400 | 4.25904 | 114 | 875 |
| 25 | 21.3791 | 4.15284 | 117 | 1094 |
| 26 | 22.6378 | 3.92470 | 147 | 1489 |
| 27 | 23.7600 | 3.74182 | 63 | 649 |
| 28 | 23.9600 | 3.71103 | 119 | 0 |
| 29 | 24.4137 | 3.64309 | 314 | 2085 |
| 30 | 25.2127 | 3.52942 | 76 | 999 |
| 31 | 26.7200 | 3.33364 | 63 | 600 |
| 32 | 27.1738 | 3.27899 | 234 | 2018 |

This unique set of XRPD peak positions or a subset thereof can be used to identify Form E. One such subset comprises peaks at about 5.1, 5.3 and 16.4 °2θ. Another subset comprises peaks at about 9.7 and 20.8 °2θ.

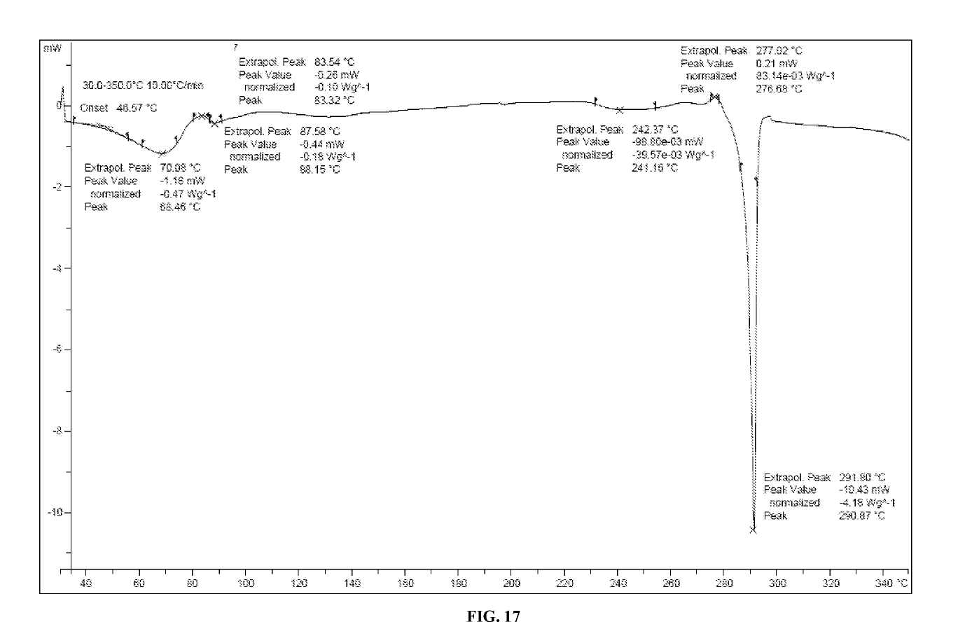

KF analysis results showed 0.8% water. A DSC thermogram of Form E (FIG. 17) showed multiple events, with endotherms at 276 and 291° C. and an exothermic event at 278° C.

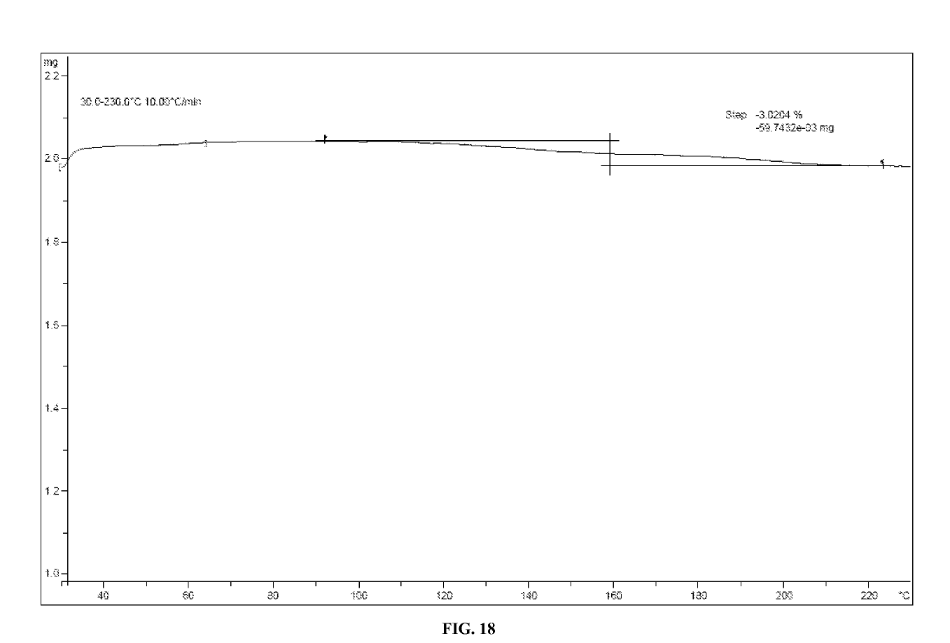

FIG. 18 is a TGA thermogram of Form E and it shows 3.0% weight loss between 100 and 220° C.

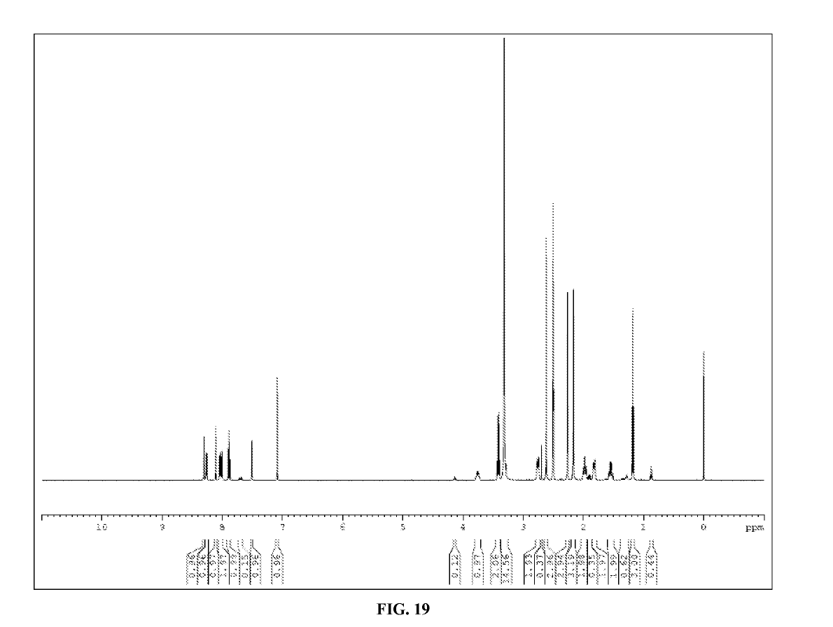

Form E was further characterized by solution ¹H NMR. The spectrum is reported in FIG. 19. The spectrum is consistent with one molar equivalent of solvent present, as well as the known chemical structure of Compound I.

Further details related to the preparation and characterization of Form E are presented below in the Examples section.

6. Form F

Based on the available characterization data, Form F appears to be an anhydrous high-melt form of Compound I that was isolated using DSC experiments. Form F was characterized by techniques including XRPD, DSC, and ¹H-NMR. Table 9 summarizes some of these results. Form F can be isolated in DSC experiments using heat-cool experiments with an isothermal hold at 280° C. as shown in Tables 17 and 18.

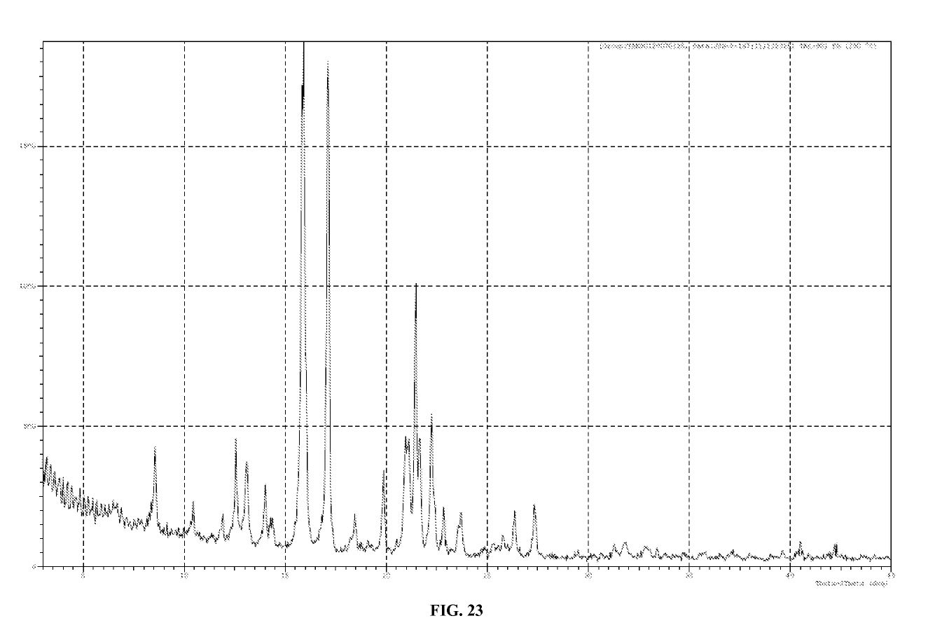

FIG. 23 shows a characteristic XRPD spectrum (CuKα, λ=1.5418 Å) of Form F. The XRPD pattern confirms that Form F is crystalline. Major X-Ray diffraction lines expressed in °2θ and their relative intensities are summarized in Table 6.

TABLE 6
Characteristic XRPD Peaks (CuKα) of Form F

| Peak No. | 2θ (°) | d-spacing | Intensity | I/Iₒ |
|---|---|---|---|---|
| 1 | 5.7161 | 15.44873 | 101 | 864 |
| 2 | 6.0400 | 14.62099 | 116 | 578 |
| 3 | 6.2400 | 14.15281 | 298 | 2032 |
| 4 | 6.6041 | 13.37332 | 2297 | 12890 |
| 5 | 8.5043 | 10.38897 | 281 | 1798 |
| 6 | 10.0000 | 8.83820 | 87 | 609 |
| 7 | 10.2641 | 8.61138 | 267 | 1745 |
| 8 | 11.2662 | 7.84757 | 265 | 1824 |
| 9 | 13.2298 | 6.68689 | 134 | 889 |
| 10 | 13.5444 | 6.53227 | 88 | 706 |
| 11 | 16.6800 | 5.31069 | 437 | 4685 |
| 12 | 17.1179 | 5.17581 | 2913 | 17588 |
| 13 | 17.6000 | 5.03511 | 192 | 1933 |
| 14 | 17.8800 | 4.95688 | 99 | 735 |
| 15 | 19.8681 | 4.46513 | 99 | 786 |

This unique set of XRPD peak positions or a subset thereof can be used to identify Form F. One such subset comprises peaks at about 6.6, 16.7 and 17.1 °2θ. Another subset comprises peaks at about 6.2 and 11.2 °2θ.

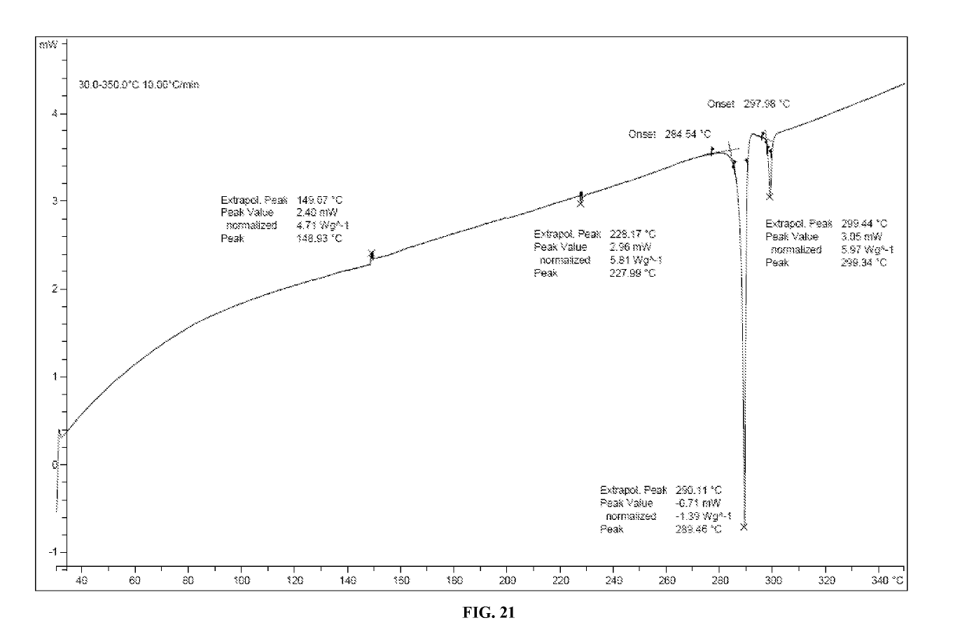

FIG. 21 shows a characteristic DSC thermogram of Form F. A first endotherm centered at about 289° C., a second endotherm centered at about 299° C., and an exotherm centered at about 149° C. were observed.

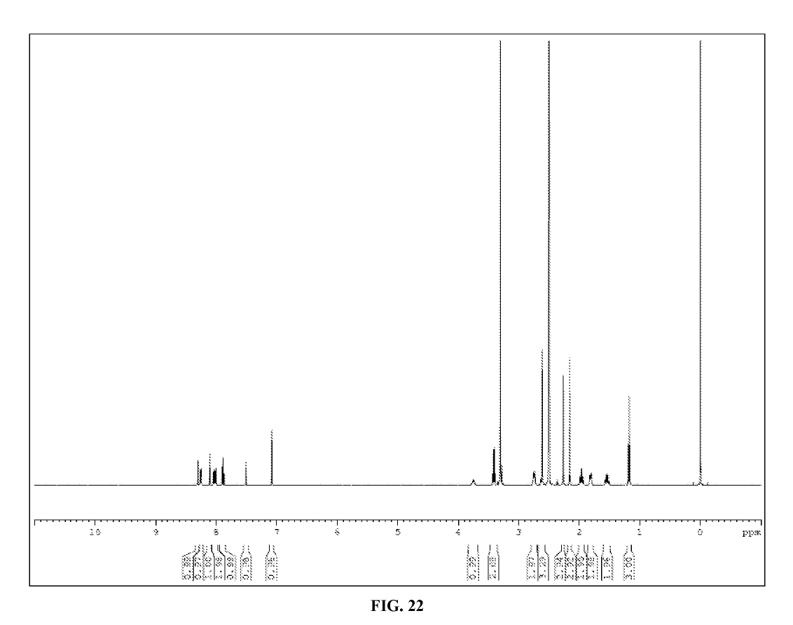

Form F was further characterized by solution ¹H NMR. The spectrum is reported in FIG. 22. Chemical assignments were not performed; however, the spectra are consistent with the known chemical structure of Compound I.

7. Form G

Based on the available characterization data, Form G appears to be an anhydrous high-melt form of Compound I that was isolated using DSC experiments. Form G was characterized by techniques including XRPD, DSC, and ¹H-NMR. Table 9 summarizes some of these results. Form G can be isolated in DSC experiments using heat-cool experiments with an isothermal hold at 290° C. as shown in Tables 17 and 18.

FIG. 23 shows a characteristic XRPD spectrum (CuKα, λ=1.5418 Å) of Form G. The XRPD pattern confirms that Form G is crystalline. Major X-Ray diffraction lines expressed in °2θ and their relative intensities are summarized in Table 7.

TABLE 7
Characteristic XRPD Peaks (CuKα) of Form G

| Peak No. | 2θ (°) | d-spacing | Intensity | I/Iₒ |
|---|---|---|---|---|
| 1 | 3.8067 | 23.19230 | 46 | 313 |
| 2 | 8.5433 | 10.34164 | 196 | 1245 |
| 3 | 10.4195 | 8.48329 | 72 | 475 |
| 4 | 11.8663 | 7.45201 | 52 | 281 |
| 5 | 12.5527 | 7.04603 | 204 | 1210 |
| 6 | 13.0714 | 6.76757 | 222 | 1239 |
| 7 | 13.9902 | 6.32511 | 138 | 640 |
| 8 | 14.3085 | 6.18511 | 67 | 429 |
| 9 | 15.8731 | 5.57880 | 1424 | 9554 |
| 10 | 16.7200 | 5.29808 | 63 | 614 |
| 11 | 17.0990 | 5.18149 | 1323 | 6504 |
| 12 | 18.4289 | 4.81046 | 86 | 636 |
| 13 | 19.8433 | 4.47066 | 195 | 971 |
| 14 | 21.0112 | 4.22472 | 304 | 2937 |
| 15 | 21.4378 | 4.14160 | 670 | 3096 |
| 16 | 21.6800 | 4.09588 | 269 | 1037 |
| 17 | 22.2349 | 3.99490 | 336 | 2024 |
| 18 | 22.8209 | 3.89363 | 99 | 424 |
| 19 | 23.6353 | 3.76127 | 105 | 695 |
| 20 | 25.7764 | 3.45350 | 56 | 455 |
| 21 | 26.3245 | 3.38282 | 120 | 650 |
| 22 | 27.3217 | 3.26158 | 138 | 797 |
| 23 | 31.7800 | 2.81346 | 45 | 525 |
| 24 | 40.4888 | 2.22614 | 44 | 288 |

This unique set of XRPD peak positions or a subset thereof can be used to identify Form G. One such subset comprises peaks at about 15.9, 17.1 and 21.4 degrees °2θ. Another subset comprises peaks at about 21.0 and 22.2 °2θ.

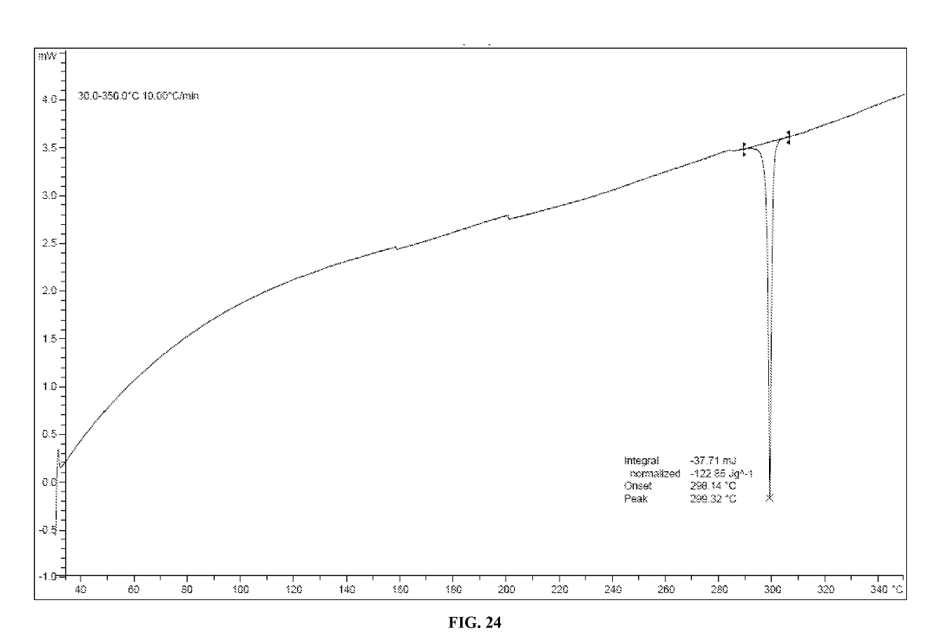

FIG. 24 shows a characteristic DSC thermogram of Form G. An endotherm was observed at approximately 299° C. (peak maximum).

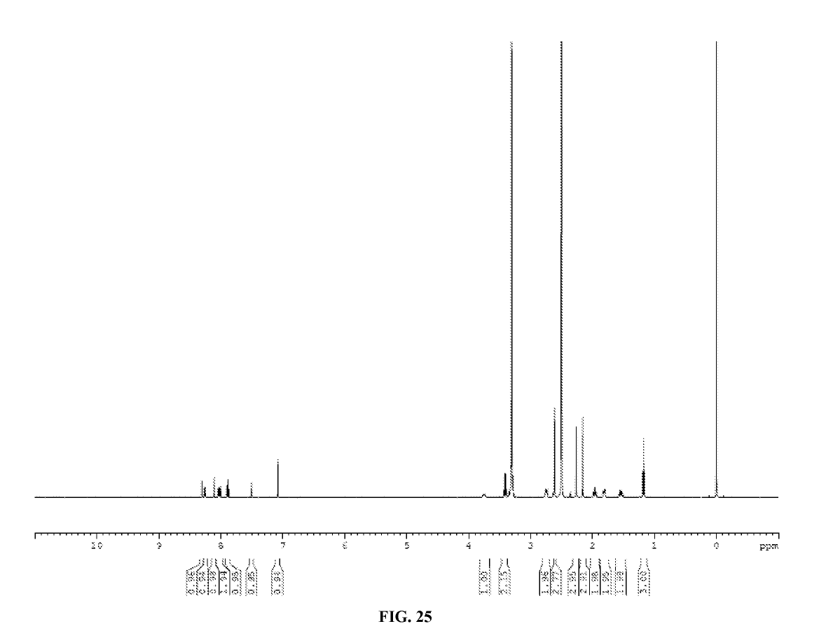

Form G was further characterized by solution ¹H NMR. The spectrum is reported in FIG. 25. The spectrum is consistent with one molar equivalent of solvent present, as well as the known chemical structure of Compound I.

Further details related to the preparation and characterization of Form G are presented below in the Examples section.

D. HPF₆ Salt of Compound I

The HPF₆ salt of Compound I was characterized by techniques including XRPD, DSC, TGA, ¹H-NMR, ¹⁹F-NMR and ³¹P-NMR. Based on the available characterization data, this appears to be an anhydrous form of a HPF₆ salt of Compound I that is stable under ambient conditions.

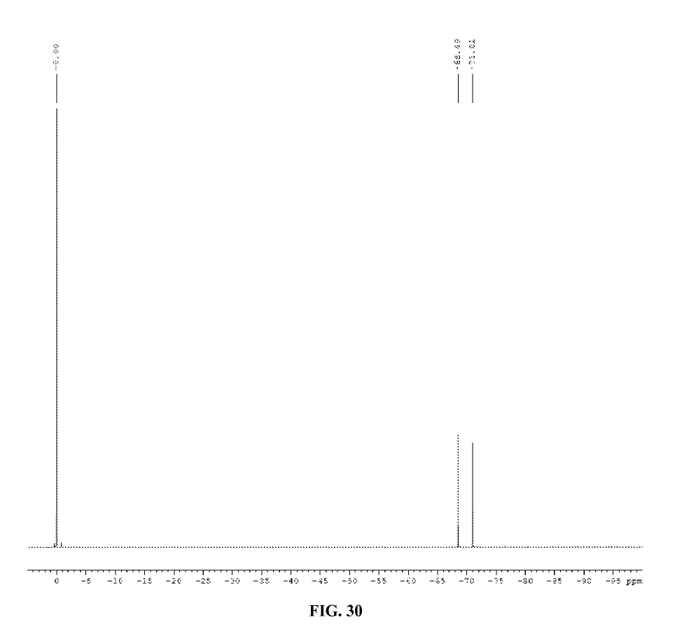

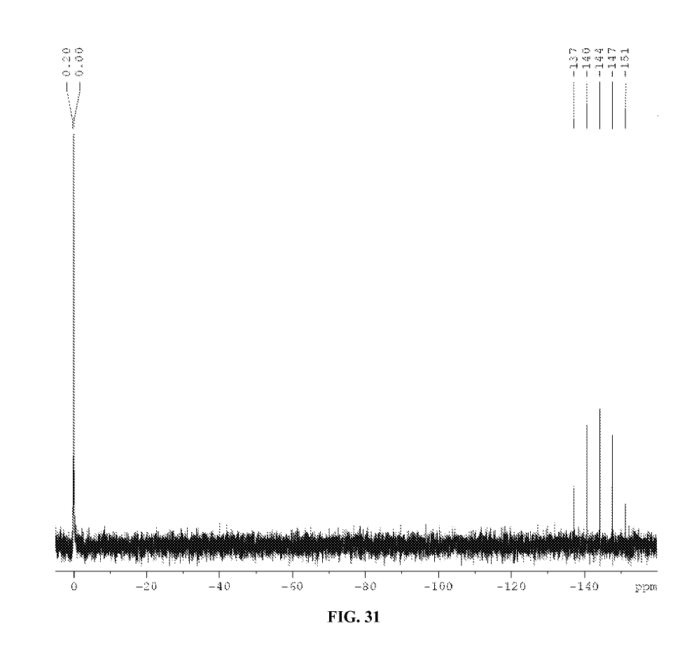

¹⁹F and ³¹P NMR analysis (FIGS. 30 and 31) for this salt form of Compound I is consistent with the expected splitting for HPF₆. Analysis by TGA showed a 2.4% weight loss between 40 and 60° C. which corresponds to the 2.1% water content observed by Karl Fisher. Phosphorus content of the HPF₆ salt Compound I, was found to be 1.5 wt %, which was below the theoretical content for a 1:1 ratio of 4.8 wt %. This deviation from the theoretical value was not unexpected due to the un-optimized nature of the process used to generate the salt as it was not the intended product.

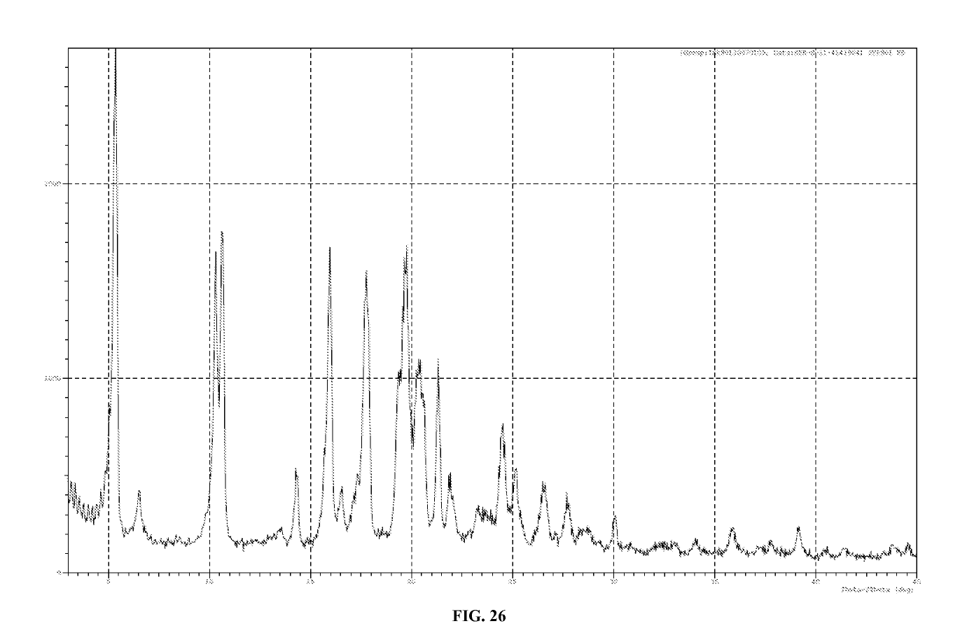

FIG. 26 shows a characteristic XRPD spectrum (CuKα, λ=1.5418 Å) of this form of the HPF₆ Salt. The XRPD pattern confirms that Form 1 is crystalline. Major X-ray diffraction lines expressed in °2θ and their relative intensities are summarized in Table 8.

TABLE 8
Characteristic XRPD Peaks (CuKα) of HPF₆ Salt of Compound I

| Peak No. | 2θ (°) | d-spacing | Intensity | I/Iₒ |
|---|---|---|---|---|
| 1 | 3.8067 | 23.19230 | 46 | 313 |
| 2 | 8.5433 | 10.34164 | 196 | 1245 |
| 3 | 10.4195 | 8.48329 | 72 | 475 |
| 4 | 11.8663 | 7.45201 | 52 | 281 |
| 5 | 12.5527 | 7.04603 | 204 | 1210 |
| 6 | 13.0714 | 6.76757 | 222 | 1239 |
| 7 | 13.9902 | 6.32511 | 138 | 640 |
| 8 | 14.3085 | 6.18511 | 67 | 429 |
| 9 | 15.8731 | 5.57880 | 1424 | 9554 |
| 10 | 16.7200 | 5.29808 | 63 | 614 |
| 11 | 17.0990 | 5.18149 | 1323 | 6504 |
| 12 | 18.4289 | 4.81046 | 86 | 636 |
| 13 | 19.8433 | 4.47066 | 195 | 971 |
| 14 | 21.0112 | 4.22472 | 304 | 2937 |
| 15 | 21.4378 | 4.14160 | 670 | 3096 |
| 16 | 21.6800 | 4.09588 | 269 | 1037 |
| 17 | 22.2349 | 3.99490 | 336 | 2024 |
| 18 | 22.8209 | 3.89363 | 99 | 424 |
| 19 | 23.6353 | 3.76127 | 105 | 695 |
| 20 | 25.7764 | 3.45350 | 56 | 455 |
| 21 | 26.3245 | 3.38282 | 120 | 650 |
| 22 | 27.3217 | 3.26158 | 138 | 797 |
| 23 | 31.7800 | 2.81346 | 45 | 525 |
| 24 | 40.4888 | 2.22614 | 44 | 288 |

This unique set of XRPD peak positions or a subset thereof can be used to identify this form of the HPF₆ Salt. One such subset comprises peaks at about 7.0, 16.7 and 17.4 °2θ. Another subset comprises peaks at about 19.6, 20.2 and 24.6 °2θ.

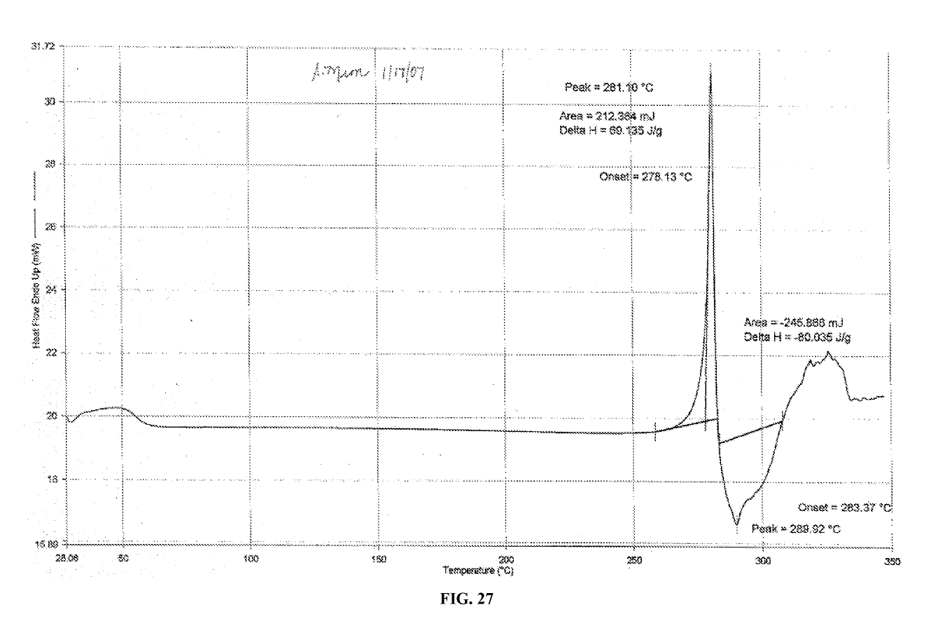

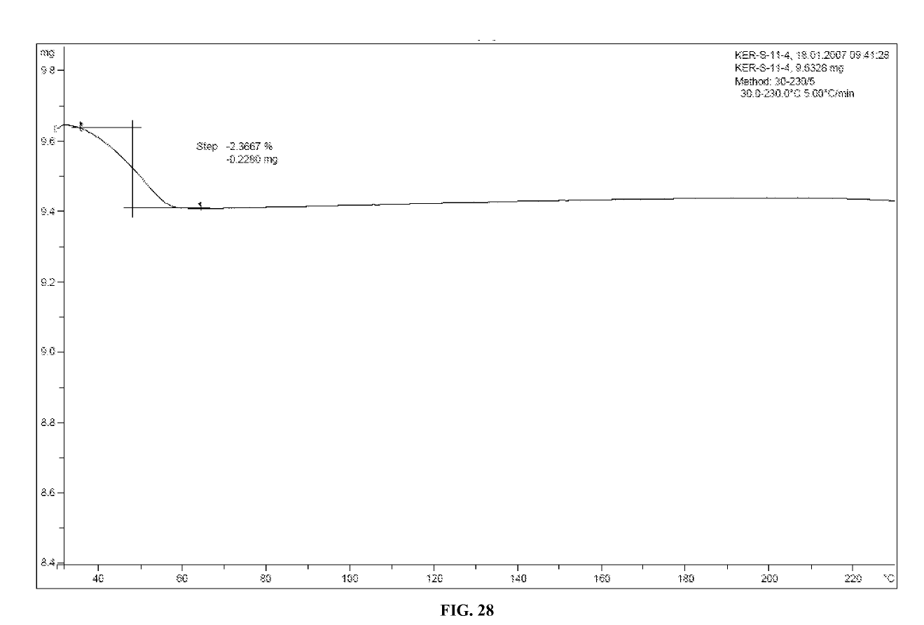

DSC analysis (FIG. 27) showed a broad endotherm near 50° C. and a single large endotherm at 281° C. This differs from the free base forms of Compound I which showed a melt/recrystallizations near 280° C. followed by a final transition near 290° C. FIG. 28 is a TGA thermogram of this form of the HPF₆ salt of Compound I.

Further details related to the preparation and characterization of the HPF₆ salt Compound I, including this solid form, are presented below in the Examples section.

### Indications for Use of Compound I

The present invention also relates to methods to alter, preferably to reduce kinase activity within a subject by administrating Compound I in a form selected from the group consisting of Form A through Form G, and pharmaceutically acceptable salts of Compound I.

Kinases are believed to contribute to the pathology and/or symptomology of several different diseases such that reduction of the activity of one or more kinases in a subject through inhibition may be used to therapeutically address these disease states. Examples of various diseases that may be treated using Compound I of the present invention are described herein. It is noted that additional diseases beyond those disclosed herein may be later identified as the biological roles that kinases play in various pathways becomes more fully understood.

Compound I may be used to treat or prevent cancer. In one embodiment, Compound I is used in a method comprising administering a therapeutically effective amount of Compound I or a composition comprising Compound I to a mammalian species in need thereof. In particular embodiments, the cancer is selected from the group consisting of squamous cell carcinoma, astrocytoma, Kaposi's sarcoma, glioblastoma, small-cell lung cancer, non small-cell lung cancers (e.g., large cell lung cancer, adenocarcinoma and squamous cell carcinoma), bladder cancer, head and neck cancer, melanoma, ovarian cancer, prostate cancer, breast cancer, glioma, colorectal cancer, genitourinary cancer, gastrointestinal cancer, thyroid cancer, skin cancer, kidney cancer, rectal cancer, colonic cancer, cervical cancer, mesothelioma, pancreatic cancer, liver cancer, uterus cancer, cerebral tumor cancer, urinary bladder cancer and blood cancers including multiple myeloma, chronic myelogenous leukemia and acute lymphocytic leukemia. In other embodiments, Compound I is useful for inhibiting growth of cancer, for suppressing metastasis of cancer, for suppressing apoptosis and the like.

In another embodiment, Compound I is used in a method for treating inflammation, inflammatory bowel disease, psoriasis, or transplant rejection, comprising administration to a mammalian species in need thereof a therapeutically effective amount of Compound I or a composition comprising Compound I.

In another embodiment, Compound I is used in a method for preventing or treating amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication, comprising administration to a mammalian species in need thereof of a therapeutically effective amount of Compound I or a composition comprising Compound I.

In yet another embodiment, Compound I is used in a method for preventing or treating mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment and androgenetic alopecia, comprising administering to a mammal, including man in need of such prevention and/or treatment, a therapeutically effective amount of Compound I or a composition comprising Compound I.

In a further embodiment, Compound I is used in a method for preventing or treating dementia related diseases, Alzheimer's Disease and conditions associated with kinases, comprising administration to a mammalian species in need thereof of a therapeutically effective amount of Compound I or a composition comprising Compound I. In one particular variation, the dementia related diseases are selected from the group consisting of Frontotemporal dementia Parkinson's Type, Parkinson dementia complex of Guam, HIV dementia, diseases with associated neurofibrillar tangle pathologies, predemented states, vascular dementia, dementia with Lewy bodies, Frontotemporal dementia and dementia pugilistica.

In another embodiment, Compound I is used in a method for treating arthritis comprising administration to a mammalian species in need thereof of a therapeutically effective amount of Compound I or a composition comprising Compound I.

Compositions, according to the present invention, may be administered, or coadministered with other active agents. These additional active agents may include, for example, one or more other pharmaceutically active agents. Coadministration in the context of this invention is intended to mean the administration of more than one therapeutic agent, one of which includes Compound I. Such co-administration may also be coextensive, that is, occurring during overlapping periods of time or may be sequential, that is, occurring during non-overlapping periods of time. Examples of co-administration of Compound I with other active ingredients in a combination therapy are described in U.S. Patent Publication No. 2007-0117816, published May 24, 2007 (see Compound 112) and U.S. Patent Application Nos. 60/912,625 and 60/912,629, filed Apr. 18, 2007 (see Compound 83), which are incorporated herein by reference in their entireties.

For oncology indications, Compound I may be administered in conjunction with other agents to inhibit undesirable and uncontrolled cell proliferation. Examples of other anti-cell proliferation agents that may be used in conjunction with Compound I include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulfate (clupeine), sulfated chitin derivatives (prepared from queen crab shells), sulfated polysaccharide peptidoglycan complex (sp-pg), staurosporine, modulators of matrix metabolism, including for example, proline analogs ((1-azetidine-2-carboxylic acid (LACA)), cishydroxyproline, d,1-3,4-dehydroproline, thiaproline, beta.-aminopropionitrile fumarate, 4-propyl-5-(4-pyridinyl)-2(3H)-oxazolone, methotrexate, mitoxantrone, heparin, interferons, 2 macroglobulin-serum, chimp-3, chymostatin, beta.-cyclodextrin tetradecasulfate, eponemycin; fumagillin, gold sodium thiomalate, d-penicillamine (CDPT), beta.-1-anticollagenase-serum, alpha.2-antiplasmin, bisantrene, lobenzarit disodium, n-2-carboxyphenyl-4-chloroanthronilic acid disodium or “CCA”, thalidomide; angostatic steroid, carboxyaminoimidazole; metalloproteinase inhibitors such as BB94. Other anti-angiogenesis agents that may be used include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

In another embodiment, a therapeutic method is provided that comprises administering Compound I. In another embodiment, a method of inhibiting cell proliferation is provided that comprises contacting a cell with an effective amount of Compound I. In another embodiment, a method of inhibiting cell proliferation in a patient is provided that comprises administering to the patient a therapeutically effective amount of Compound I.

In another embodiment, a method of treating a condition in a patient which is known to be mediated by one or more kinases, or which is known to be treated by kinase inhibitors, comprising administering to the patient a therapeutically effective amount of Compound I. In another embodiment, a method is provided for using Compound I in order to manufacture a medicament for use in the treatment of a disease state which is known to be mediated by one or more kinases, or which is known to be treated by kinase inhibitors.

In another embodiment, a method is provided for treating a disease state for which kinases possess activity that contributes to the pathology and/or symptomology of the disease state, the method comprising: administering Compound Ito a subject such that the free base form of Compound I is present in the subject in a therapeutically effective amount for the disease state.

The present invention relates generally to a method comprising administering between 1 mg/day and 500 mg/day of Compound Ito a patient, optionally between 1 mg/day and 400 mg/day of Compound I, optionally between 1 mg/day and 250 mg/day of Compound I, optionally between 2.5 mg/day and 200 mg/day of Compound I, optionally between 2.5 mg/day and 150 mg/day of Compound I, and optionally between 5 mg/day and 100 mg/day of Compound I (in each instance based on the molecular weight of the free base form of Compound I). Specific dosage amounts that may be used include, but are not limited to 2.5 mg, 5 mg, 6.25 mg, 10 mg, 12.5 mg, 20 mg, 25 mg, 50 mg, 75 mg, 100 mg, 200 mg, 250 mg, 400 mg and 500 mg of Compound I per day. It is noted that the dosage may be administered as a daily dose or weekly dose, once daily or multiple doses per day. It is noted that Compound I may be administered in a form selected from the group consisting of Form A through Form G. However, the dosage amounts and ranges provided herein are always based on the molecular weight of the free base form of Compound I.

Compound I may be administered by any route of administration. In particular embodiments, however, the method of the present invention is practiced by administering Compound I orally.

### Pharmaceutical Compositions Comprising Compound I Where at Least One of Form A Through Form G is Present

Compound I may be used in various pharmaceutical compositions where at least a portion of Compound I is present in the composition in a form selected from the group consisting of Form A through Form G. The pharmaceutical composition should contain a sufficient quantity of Compound Ito reduce kinase activity in vivo sufficiently to provide the desired therapeutic effect. Such pharmaceutical compositions may comprise Compound I present in the composition in a range of between 0.005% and 100% (weight/weight), optionally 0.1-95%, and optionally 1-95%.

In particular embodiments, the pharmaceutical compositions comprise at least 0.1%, 0.25%, 0.5%, 1%, 5%, 10%, 25%, 50%, 75%, 80%, 85%, 90%, 95%, 97%, or 99% of Compound I in a form selected from the group consisting of Form A, Form B, Form C, Form D, Form E, Form F, Form G, and mixtures thereof In another embodiment, a particular polymorphic form selected from the group consisting of Form A, Form B, Form C, Form D, Form E, Form F, Form G, and mixtures thereof may comprise at least 0.1%, 0.25%, 0.5%, 1%, 5%, 10%, 25%, 50%, 75%, 80%, 85%, 90%, 95%, 97%, or 99% of the total amount of Compound I (weight/weight) in the pharmaceutical composition.

In addition to Compound I, the pharmaceutical composition may comprise one or more additional components that do not deleteriously affect the use of Compound I. For example, the pharmaceutical compositions may include, in addition to Compound I, conventional pharmaceutical carriers; excipients; diluents; lubricants; binders; wetting agents; disintegrating agents; glidants; sweetening agents; flavoring agents; emulsifying agents; solubilizing agents; pH buffering agents; perfuming agents; surface stabilizing agents; suspending agents; and other conventional, pharmaceutically inactive agents. In particular, the pharmaceutical compositions may comprise lactose, mannitol, glucose, sucrose, dicalcium phosphate, magnesium carbonate, sodium saccharin, carboxymethylcellulose, magnesium stearate, calcium stearate, sodium crosscarmellose, talc, starch, natural gums (e.g., gum acaciagelatin), molasses, polyvinylpyrrolidine, celluloses and derivatives thereof, povidone, crospovidones acetate, sodium citrate, cyclodextrine derivatives, sorbitan monolaurate, triethanolamine sodium acetate, triethanolamine oleate, biocompatible polymers, such as collagen, ethylene vinyl acetate, polyanhydrides, polyglycolic acid, polyorthoesters, polylactic acid and others such agents.

Pharmaceutical compositions according to the present invention may be adapted for administration by any of a variety of routes. For example, pharmaceutical compositions according to the present invention can be administered orally, parenterally, intraperitoneally, intravenously, intraarterially, topically, transdermally, sublingually, intramuscularly, rectally, transbuccally, intranasally, liposomally, via inhalation, vaginally, intraoccularly, via local delivery (for example, by catheter or stent), subcutaneously, intraadiposally, intraarticularly, or intrathecally, optionally in a slow release dosage form. In particular embodiments, the pharmaceutical compounds are administered orally, by inhalation or by injection subcutaneously, intramuscularly, intravenously or directly into the cerebrospinal fluid.

In general, the pharmaceutical compositions of the present invention may be prepared in a gaseous, liquid, semi-liquid, gel, or solid form, and formulated in a manner suitable for the route of administration to be used.

Compositions according to the present invention are optionally provided for administration to humans and animals in unit dosage forms or multiple dosage forms, such as tablets, capsules, pills, powders, dry powders for inhalers, granules, sterile parenteral solutions or suspensions, oral solutions or suspensions, oil-water emulsions, sustained release formulations, such as, but not limited to, implants and microencapsulated delivery systems, containing suitable quantities of Compound I. Methods of preparing such dosage forms are known in the art, and will be apparent to those skilled in this art; for example, see Remington's Pharmaceutical Sciences, 19^{th} Ed. (Easton, Pa.: Mack Publishing Company, 1995).

Unit-dose forms, as used herein, refers to physically discrete units suitable for human and animal subjects and packaged individually as is known in the art. Each unit-dose contains a predetermined quantity of Compound I sufficient to produce the desired therapeutic effect, in association with a pharmaceutical carrier, vehicle or diluent. Examples of unit-dose forms include ampoules and syringes, and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules, or bottles of pints or gallons. Hence, multiple dose form may be viewed as a multiple of unit-doses that are not segregated in packaging.

In general, the total amount of Compound I in a pharmaceutical composition according to the present invention should be sufficient to provide a desired therapeutic effect. This amount may be delivered as a single per day dosage, multiple dosages per day to be administered at intervals of time, or as a continuous release dosage form. Compound I may advantageously be used when administered to a patient at a daily dose of between 1 mg/day and 250 mg/day of Compound I, optionally between 2.5 mg and 200 mg of Compound I, optionally between 2.5 mg and 150 mg of Compound I, and optionally between 5 mg and 100 mg of Compound I (in each instance based on the molecular weight of the free base form of Compound I). Specific dosage amounts that may be used include, but are not limited to 2.5 mg, 5 mg, 6.25 mg, 10 mg, 12.5 mg, 20 mg, 25 mg, 50 mg, 75 mg, and 100 mg of Compound I per day. It may be desirable for Compound Ito be administered one time per day. Accordingly, pharmaceutical compositions of the present invention may be in the form of a single dose form comprising between 1 mg/day and 250 mg/day of Compound I, optionally between 2.5 mg and 200 mg of Compound I, optionally between 2.5 mg and 150 mg of Compound I, and optionally between 5 mg and 100 mg of Compound I. In specific embodiments, the pharmaceutical composition comprises 2.5 mg, 5 mg, 6.25 mg, 10 mg, 12.5 mg, 20 mg, 25 mg, 50 mg, 75 mg or 100 mg of Compound I.

A. Formulations for Oral Administration

Oral pharmaceutical dosage forms may be as a solid, gel or liquid where at least a portion of Compound I is present in the composition in a form selected from the group consisting of Form A, Form B, Form C, Form D, Form E, Form F and Form G.

In certain embodiments, Compound I is provided as solid dosage forms. Examples of solid dosage forms include, but are not limited to pills, tablets, troches, capsules, granules, and bulk powders. More specific examples of oral tablets include compressed, chewable lozenges, troches and tablets that may be enteric-coated, sugar-coated or film-coated. Examples of capsules include hard or soft gelatin capsules. Granules and powders may be provided in non-effervescent or effervescent forms. The powders may be prepared by lyophilization or by other suitable methods.

The tablets, pills, capsules, troches and the like may optionally contain one or more of the following ingredients, or compounds of a similar nature: a binder; a diluent; a disintegrating agent; a lubricant; a glidant; a coloring agent; a sweetening agent; a flavoring agent; and a wetting agent.

Examples of binders that may be used include, but are not limited to, microcrystalline cellulose, gum tragacanth, glucose solution, acacia mucilage, gelatin solution, sucrose and starch paste.

Examples of diluents that may be used include, but are not limited to, lactose, sucrose, starch, kaolin, salt, mannitol and dicalcium phosphate.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of lubricants that may be used include, but are not limited to, talc, starch, magnesium or calcium stearate, lycopodium and stearic acid.

Examples of glidants that may be used include, but are not limited to, colloidal silicon dioxide.

Examples of coloring agents that may be used include, but are not limited to, any of the approved certified water soluble FD and C dyes, mixtures thereof; and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

When the dosage form is a pill, tablet, torches, or the like, Compound I may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compound I may also be administered as a component of an elixir, emulsion, suspension, microsuspension, syrup, wafer, sprinkle, chewing gum or the like. A syrup may optionally comprise, in addition to the active compounds, sucrose as a sweetening agent and certain preservatives, dyes and colorings and flavors.

Alternatively, liquid or semi-solid oral formulations may be prepared by dissolving or dispersing the active compound or salt in vegetable oils, glycols, triglycerides, propylene glycol esters (e.g. propylene carbonate) and other such carriers, and encapsulating these solutions or suspensions in hard or soft gelatin capsule shells. Other useful formulations include those set forth in U.S. Pat. Nos. Re 28,819 and 4,358,603.

Examples of oral formulations that may be used to administer Compound I has been described in U.S. patent application Ser. No. 11/531,671, filed Sep. 13, 2006, the disclosure of which is herein expressly incorporated by reference in its entirety.

Exemplary tablet formulations are provided below. It is noted that the examples are, by way of illustration but not limitation. It is also noted that Compound I is present in the formulation in a form selected from the group consisting of one or more of Form A, Form B, Form C, Form D, Form E, Form F, and Form G. It is also noted that the formulations provided herein may be varied as is known in the art.

### 12.5 mg of Compound I (Weight of Free Base Form) Per Tablet

### Core Tablet Formulation

| | | |
|---|---|---|
| (1) | Compound I | 17.0 mg |
| (2) | Lactose Monohydrate, NF, Ph, Eur | 224.6 mg |
| | (FOREMOST 316 FAST FLO) | |
| (3) | Microcrystalline Cellulose, NF, Ph, Eur | 120.1 mg |
| | (AVICEL PH 102) | |
| (4) | Crosscarmellose Sodium, NF, Ph, Eur | 32.0 mg |
| | (AC-DO-SOL) | |
| (5) | Colloidal Silicon Dioxide, NF, Ph, Eur | 3.2 mg |
| | (CAB-O-SIL M-5P) | |
| (6) | Magnesium Stearate, NF, Ph, Eur | 3.2 mg |
| | (MALLINCKRODT, Non-bovine Hyqual) | |

| | |
|---|---|
| TOTAL (per tablet) | 400.0 mg |

### Film Coat (12.0 mg in Total)

(1) Opadry II 85F18422, White—Portion 1 (COLORCON)
(2) Opadry II 85F18422, White—Portion 2 (COLORCON)
(3) Opadry II 85F18422, White—Portion 3 (COLORCON)

### 25 mg of Compound I (Weight of Free Base Form) Per Tablet

### Core Tablet Formulation

| | | |
|---|---|---|
| (1) | Compound I | 34.0 mg |
| (2) | Lactose Monohydrate, NF, Ph, Eur | 207.6 mg |
| | (FOREMOST 316 FAST FLO) | |
| (3) | Microcrystalline Cellulose, NF, Ph, Eur | 120.1 mg |
| | (AVICEL PH 102) | |
| (4) | Crosscarmellose Sodium, NF, Ph, Eur | 32.0 mg |
| | (AC-DO-SOL) | |
| (5) | Colloidal Silicon Dioxide, NF, Ph, Eur | 3.2 mg |
| | (CAB-O-SIL M-5P) | |
| (6) | Magnesium Stearate, NF, Ph, Eur | 3.2 mg |
| | (MALLINCKRODT, Non-bovine Hyqual) | |

| | |
|---|---|
| TOTAL (per tablet) | 400.0 mg |

### Film Coat (12.0 mg in Total)

(1) Opadry II 85F18422, White—Portion 1 (COLORCON)
(2) Opadry II 85F18422, White—Portion 2 (COLORCON)
(3) Opadry II 85F18422, White—Portion 3 (COLORCON)

### 50 mg of Compound I (Weight of Free Base Form) Per Tablet

### Core Tablet Formulation

| | | |
|---|---|---|
| (1) | Compound I | 68.0 mg |
| (2) | Lactose Monohydrate, NF, Ph, Eur | 173.6 mg |
| | (FOREMOST 316 FAST FLO) | |
| (3) | Microcrystalline Cellulose, NF, Ph, Eur | 120.1 mg |
| | (AVICEL PH 102) | |
| (4) | Crosscarmellose Sodium, NF, Ph, Eur | 32.0 mg |
| | (AC-DO-SOL) | |
| (5) | Colloidal Silicon Dioxide, NF, Ph, Eur | 3.2 mg |
| | (CAB-O-SIL M-5P) | |
| (6) | Magnesium Stearate, NF, Ph, Eur | 3.2 mg |
| | (MALLINCKRODT, Non-bovine Hyqual) | |

| | |
|---|---|
| TOTAL (per tablet) | 400.0 mg |

### Film Coat (12.0 mg in Total)

(1) Opadry II 85F18422, White—Portion 1 (COLORCON)
(2) Opadry II 85F18422, White—Portion 2 (COLORCON)
(3) Opadry II 85F18422, White—Portion 3 (COLORCON)

B. Injectables, Solutions and Emulsions

Compound I present in a form or a mixture of forms selected from the group consisting of Form A, Form B, Form C, Form D, Form E, Form F, and Form G may be formulated for parenteral administration. Parenteral administration generally characterized by injection, either subcutaneously, intramuscularly or intravenously. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the route of administration and the indication of disease to be treated.

Injectables may be prepared in any conventional form. These formulations include, but are not limited to, sterile solutions, suspensions, microsuspensions, and emulsions ready for injection, and solid forms, e.g., lyophilized or other powders including hypodermic tablets, ready to be combined with a carrier just prior to use. Generally, the resulting formulation may be a solution, microsuspension, suspension and emulsion. The carrier may be an aqueous, non-aqueous liquid, or a solid vehicle that can be suspended in liquid.

Examples of carriers that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions includes EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of Compound I in the parenteral formulation may be adjusted so that an injection administers a pharmaceutically effective amount sufficient to produce the desired pharmacological effect. The exact concentration of Compound I and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is known and practiced in the art.

Injectables may be designed for local and systemic administration. Typically a therapeutically effective dosage is formulated to contain a concentration of at least about 0.1% w/w up to about 90% w/w or more, preferably more than 1% w/w of Compound Ito the treated tissue(s). Compound I may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

Compound I may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

C. Powders

Compound I in a form or a mixture of forms selected from the group consisting of one or more of Form A, Form B, Form C, Form D, Form E, Form F, and Form G may be prepared as powders, which can be reconstituted for administration as solutions, emulsions and other mixtures. The powders may also be formulated as solids or gels.

Powders of Compound I may be prepared by grinding, spray drying, lyophilization and other techniques that are well known in the art. Sterile, lyophilized powder may be prepared by dissolving Compound I in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, Compound I is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of Compound I.

D. Topical Administration

Compound I present in a form or a mixture of forms selected from the group consisting of Form A, Form B, Form C, Form D, Form E, Form F, and Form G may also be administered as topical mixtures. Topical mixtures may be used for local and systemic administration. The resulting mixture may be a solution, suspension, microsuspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

Compound I may be formulated for topical applications to the respiratory tract. These pulmonary formulations can be in the form of an aerosol, solution, emulsion, suspension, microsuspension for a nebulizer, or as a microfine powder for insufflation, alone or in combination with an inert carrier such as lactose. In such a case, the particles of the formulation will typically have diameters of less than 50 microns, preferably less than 10 microns. Examples of aerosols for topical application, such as by inhalation are disclosed in U.S. Pat. Nos. 4,044,126, 4,414,209, and 4,364,923, which describe aerosols for delivery of a steroid useful for treatment inflammatory diseases, particularly asthma.

Compound I may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions or suspensions of Compound I alone or in combination with other pharmaceutically acceptable excipients can also be administered.

E. Formulations for Other Routes of Administration

Depending upon the disease state being treated, Compound I present in a form or a mixture of forms selected from the group consisting of Form A, Form B, Form C, Form D, Form E, Form F, and Form G may be formulated for other routes of administration, such as topical application, transdermal patches, and rectal administration. For example, pharmaceutical dosage forms for rectal administration are rectal suppositories, capsules and tablets for systemic effect. Rectal suppositories as used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

### Kits and Articles of Manufacture Comprising Compound I Polymorphs

The present invention is also directed to kits and other articles of manufacture for treating diseases associated with kinases. It is noted that diseases are intended to cover all conditions for which kinases possess activity that contributes to the pathology and/or symptomology of the condition.

In one embodiment, a kit is provided that comprises a pharmaceutical composition comprising Compound I where greater than 0.1%, 0.25%, 0.5%, 1%, 5%, 10%, 25%, 50%, 75%, 80%, 85%, 90%, 95%, 97% or 99% of Compound I (by weight) is present in a form selected from the group consisting of Form A, Form B, Form C, Form D, Form E, Form F, and Form G; and instructions for use of the kit. Optionally, the composition comprises at least 0.1%, 0.25%, 0.5%, 1%, 5%, 10%, 25%, 50%, 75%, 80%, 85%, 90%, 95%, 97%, or 99% of Compound I. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a pharmaceutical composition comprising Compound I where greater than 0.1%, 0.25%, 0.5%, 1%, 5%, 10%, 25%, 50%, 75%, 80%, 85%, 90%, 95%, 97% or 99% of Compound I (by weight) is present in the composition in a form selected from the group consisting of Form A, Form B, Form C, Form D, Form E, Form F, and Form G; and packaging materials. Optionally, the composition comprises at least 0.1%, 0.25%, 0.5%, 1%, 5%, 10%, 25%, 50%, 75%, 80%, 85%, 90%, 95%, 97%, or 99% of Compound I. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLES

### Example 1

### Preparation of 5-(3-(ethylsulfonyl)phenyl)-3,8-dimethyl-N-(1-methylpiperidin-4-yl)-9h-pyrido[2,3-b]indole-7-carboxamide (Compound I)

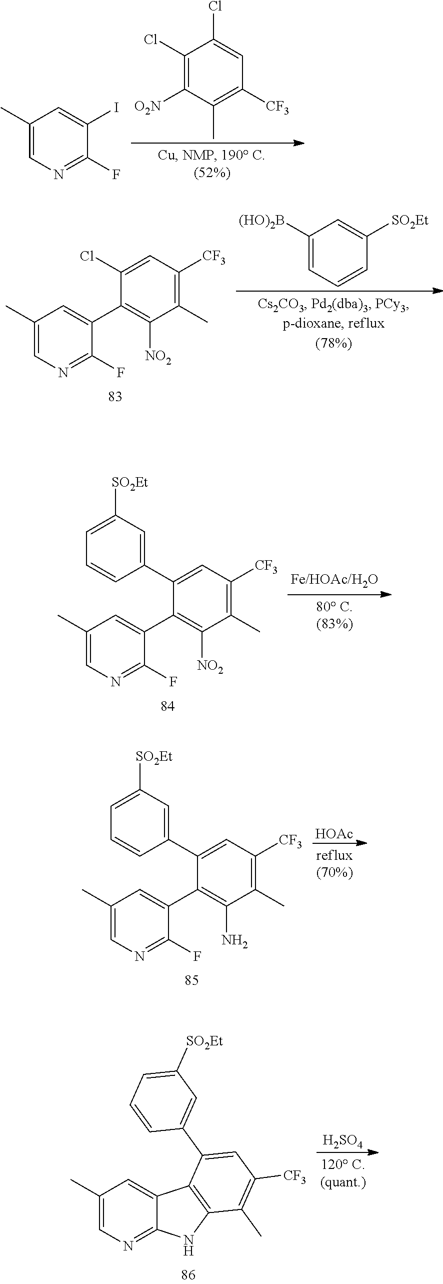

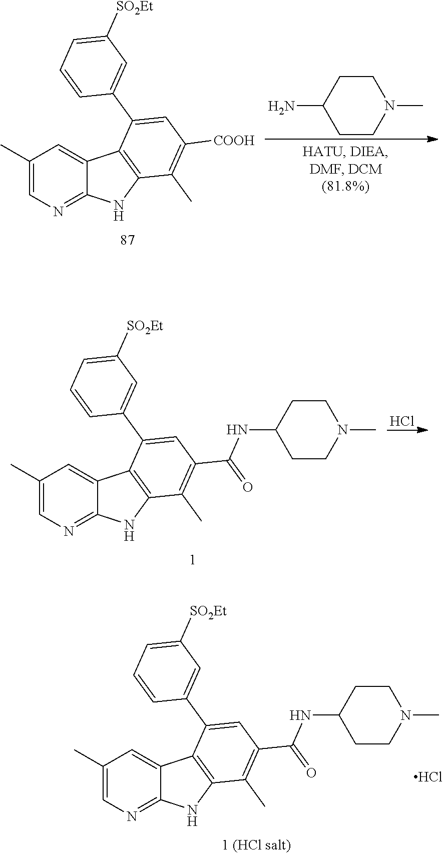

3-(6-chloro-3-methyl-2-nitro-4-(trifluoromethyl)phenyl)-2-fluoro-5-methylpyridine: 2-Fluoro-3-iodo-5-picoline (15.0 g, 63 mmol) was added drop wise during 2 h as a solution in NMP (20 mL) to a stirred suspension of 3,4-dichlororo-2-nitro-6-(trifluoromethyl)-toluene (52.1 g, 190 mmol) and copper (12.1 g, 190 mmol) in NMP (115 mL) at 190° C. After completion of the reaction (2.5 h), the mixture was cooled to room temperature, filtered, rinsed with NMP (3×5 mL) followed by EtOAc (1×100 mL). The filtrate was diluted with EtOAc (400 mL) affording a turbid solution. The organic layer was partitioned with sat. NaHCO₃ (150 mL) affording a suspension/emulsion. H₂O (50 mL) and MeOH (50 mL) were added to aid solubility. The aqueous layer was washed with EtOAc (5×150 mL). The organic layers were combined, dried (MgSO₄), and concentrated in vacuo. The crude product was purified by silica gel chromatography (98:2 Toluene:EtOAc) to provide the title compound as a tan solid (11.4 g, 52%). ¹H NMR (400 MHz, DMSO-d₆): δ 8.34 (s, 1H), 8.26 (s, 1H), 7.86-7.89 (m, 1H), 2.4 (s, 3H), 2.34 (s, 3H). MS (ES) [m+H] calc'd for C₁₄H₉ClF₄N₂O₂, 349; found 349.2.

3-(3′-(ethylsulfonyl)-4-methyl-3-nitro-5-(trifluoromethyl)biphenyl-2-yl)-2-fluoro-5-methylpyridine: A mixture of Compound 83 (6.0 g, 17.2 mmol), 3-ethylsulfonylphenylboronic acid (4.79 g, 22.4 mmol), bis(dibenzylideneacetone)Pd(0) (1.48 g, 2.6 mmol), tricyclohexylphosphine (1.45 g, 5.2 mmol), Cs₂CO₃ (14.0 g, 43 mmol), and dioxane (60 mL) was heated at reflux for 4.5 hr. After completion the reaction was cooled to room temperature, filtered, rinsed with dioxane, and concentrated in vacuo. The resulting oil was reconstituted in EtOAc (75 mL) washed with H₂O (1×30 mL) and brine (1×30 mL), dried (MgSO₄), and concentrated in vacuo. The crude product was purified by silica gel chromatography (4:1 hexanes/EtOAc) to provide the title compound as a tan solid (6.5 g, 78%). ¹H NMR (400 MHz, DMSO-d₆): δ 8.15 (s, 1H), 8.04 (s, 1H), 7.90-7.93 (m, 1H), 7.80-7.82 (m, 1H), 7.60-7.70 (m, 3H), 3.1-3.2 (m, 2H), 2.49 (s, 3H), 2.25 (s, 3H), 0.85 (t, 3H). MS (ES) [m+H] calc'd for C₂₂H₁₈F₄N₂O₄S, 483; found 483.3.

3′-(ethylsulfonyl)-2-(2-fluoro-5-methylpyridin-3-yl)-4-methyl-5-(trifluoromethyl)biphenyl-3-amine: A mixture of Compound 84 (6.4 g, 13.3 mmol), iron (3.7 g, 66.3 mmol), HOAc, (32 mL), and H₂O (11 mL) was heated at 80° C. for 2 h. After completion the reaction was concentrated in vacuo. The residue was reconstituted in dichloromethane (100 mL), filtered, and rinsed with dichloromethane (3×30 mL). The organic phase was washed with sat. NaHCO₃ (1×100 mL) and brine (1×50 mL), dried (MgSO₄), filtered, and concentrated in vacuo. The crude product was purified by silica gel chromatography (1:1 hexanes/EtOAc) to provide the title compound as a tan solid (5.0 g, 83%). ¹H NMR (400 MHz, DMSO-d₆): δ 7.93 (s, 1H), 7.67-7.7.71 (m, 2H), 7.53 (t, 1H), 7.46-7.48 (m, 1H), 7.42 (s, 1H), 6.93 (s, 1H), 5.09 (s, 2H), 3.11 (q, 2H), 2.27 (s, 3H), 2.21 (s, 3H), 0.85 (t, 3H). MS (ES) [m+H] calc'd for C₂₂H₂₀F₄N₂O₂S, 453; found 453.3.

5-(3-(ethylsulfonyl)phenyl)-3,8-dimethyl-7-(trifluoromethyl)-9H-pyrido[2,3-b]indole acetate: Compound 85 (4.9 g, 10.8 mmol) was dissolved in HOAc (35 mL) and heated at reflux for 3 h. The reaction mixture was cooled to room temperature affording a crystalline product. The resulting suspension was filtered, rinsed with HOAc (3×5 mL) followed by H₂O (3×10 mL) and the solids dried in vacuo to provide the title compound as a white solid (3.73 g, 70%). NMR analysis confirmed that the product was isolated as the mono-acetate salt. ¹H NMR (400 MHz, DMSO-d₆): δ 12.35 (s, 1H), 12.0 (s, 1H), 8.39 (s, 1H), 8.15 (s, 1H), 8.04-8.09 (m, 2H), 7.90 (t, 1H), 7.51 (s, 1H), 7.42 (s, 1H), 3.43 (q, 2H), 2.76 (s, 3H), 2.28 (s, 3H), 1.91 (s, 3H), 1.18 (t, 3H). MS (ES) [m+H] calc'd for C₂₂H₁₉F₃N₂O₂S, 433; found 433.3.

5-(3-(ethylsulfonyl)phenyl)-3,8-dimethyl-9H-pyrido[2,3-b]indole-7-carboxylic acid: Compound 86 (3.6 g, 7.3 mmol) was dissolved in concentrated H₂SO₄ (30 mL) and heated at 120° C. for 30 min. The reaction was cooled to room temperature and poured over ice affording a white precipitate. The resulting suspension was filtered, rinsed with H₂O (3×30 mL) followed by IPA (3×10 mL) and dried in vacuo to provide the title compound as a white solid (3.2 g, quant.). ¹H NMR (400 MHz, DMSO-d₆): δ 12.20 (s, 1H), 8.36 (s, 1H), 8.12 (s, 1H), 8.02-8.07 (m, 2H), 7.89 (t, 1H), 7.61 (s, 1H), 7.54 (s, 1H), 3.43 (q, 2H), 2.85 (s, 3H), 2.28 (s, 3H), 1.18 (t, 3H). MS (ES) [m+H] calc'd for C₂₂H₂₀N₂O₄S, 409; found 409.3.

5-(3-(ethylsulfonyl)phenyl)-3,8-dimethyl-N-(1-methylpiperidin-4-yl)-9H-pyrido[2,3-b]indole-7-carboxamide: A mixture of Compound 87 (11.3 g, 27.6mmol), 1-methylpiperidin-4-amine (9.47 g, 82.9 mmol), HATU (13.66 g, 35.9 mmol), DIEA (17.88 g, 138 mmol), DMF (250 mL), and DCM (250 mL) was stirred at room temperature for 30 minutes. The resulting suspension was filtered, rinsed with DMF (10 mL×4) and concentrated in vacuo. The residue was dissolved in DMSO (77 mL), filtered, and the filtrate was purified by preparative HPLC (ACN/H₂O with TFA). Following HPLC purification, the pure fractions were combined, basified with sodium bicarbonate and concentrated in vacuo to half volume. The resulting suspension was filtered, rinsed with H₂O (200 mL×5) and dried in vacuo to provide Compound I as a white solid (11.41 g, 81.8%).

The hydrochloride salt of Compound I was prepared as follows. To a stirred suspension of Compound I (8.7 g) in ACN (175 mL) and H₂O (175 mL) was added 1N HCl (18.1 mL, 1.05 eq) affording a yellow solution. After 15 minutes, the solution was frozen on dry ice/acetone and lyophilized to provide 5-(3-(ethylsulfonyl)phenyl)-3,8-dimethyl-N-(1-methylpiperidin-4-yl)-9H-pyrido[2,3-b]indole-7-carboxamide hydrochloride as a yellow solid (9.02 g, 96.7%).

The crystalline hydrochloride salt of Compound I was prepared as follows. To a stirred suspension of Compound I (0.55 g) in IPA (2.5 mL) and H₂O (2.5 mL) was added 12.1N HCl (1.05-1.10 eq) affording a yellow solution. After stirring for 45 minutes, crystallization occurred and additional IPA (15 mL) was added at room temperature. The resulting suspension was allowed to stir overnight. The solids were isolated by filtration and dried in vacuo at 60° C. to provide 5-(3-(ethylsulfonyl)phenyl)-3,8-dimethyl-N-(1-methylpiperidin-4-yl)-9H-pyrido[2,3-b]indole-7-carboxamide hydrochloride as a tan to gold colored solid (0.51 g, 87%).

Compound I was prepared from the HCl salt of Compound I by dissolving 2.52 g of the HCl salt of Compound I with stirring in 100 mL of MeCN: water (1:1, v/v). This mixture was filtered to remove a small amount of undissolved particles. To clarify the solution solid NaHCO₃ (1.99 g, 5.0 equiv.) was added in one portion followed by additional stirring at ambient temperature for 15 minutes. The suspension was concentrated to about half volume by rotary evaporation, and the resultant solution filtered and washed with water (2×25 mL). The filter cake was dried at ambient temperature under vacuum (30 inches Hg) for 24 hours to afford 2.12 g of Compound I (94.3% yield).

### Example 2

### Sample Characterization

The following analytical techniques and combination thereof were used determine the physical properties of the solid phases prepared.

1. Instrumentation

| Instrument | Vendor/Model # |
|---|---|
| Differential Scanning Calorimeter | Mettler 822^{e} DSC |
| Thermal Gravimetric Analyzer | Mettler 851^{e} SDTA/TGA |
| X-Ray Powder Diffraction System | Shimadzu XRD-6000 |
| Karl Fischer | Metrohm 756 KF Coulometer |
| Nuclear Magnetic Resonance | 500 MHz Bruker AVANCE with 5- |
| Spectrometer | mm Broadband Probe |
| High-Performance Liquid | Varian ProStar |
| Chromatography | |
| Moisture-Sorption Analysis | Hiden IGAsorp Moisture Sorption |
| | Instrument |

2. Differential Scanning Calorimetry Analysis (DSC)

Differential scanning calorimetry (DSC) analyses were carried out on samples weighed in an aluminum pan, covered with a pierced lid, and then crimped. Analysis conditions were 30° C. to 300-350° C. ramped at 10° C./min.

Samples were subjected to heat-cool-heat experiments to determine if a unique form or interconversion could be observed. Compound I was prepared by thoroughly mixing Form A lots with stirring by spatula and tumbling the powders for several minutes in a scintillation vial. Experiments were setup as described in Tables 17 and 18 with isothermal holds for five minutes at temperatures slightly below/above the observed transitions to encourage conversion/ripening. Similarly, controlled cooling profiles (−5° C./min) were used to help promote crystal growth as opposed to quench cooling which may result in amorphous or kinetically favored forms instead of the stable form.

3 Thermal Gravimetric Analysis (TGA)

Thermal gravimetric analysis (TGA) analyses were carried out on samples weighed in an alumina crucible and analyzed from 30° C. to 230° C. with a ramp rate of 10° C./min.

4. X-Ray Powder Diffraction (XRPD)

Samples for X-ray powder diffraction (XRPD) were placed on Si zero-return ultra-micro sample holders and analyzed using the following conditions:

| | |
|---|---|
| X-ray tube: | Cu Kα, 40 kV, 40 mA |
| Slits | |
| Divergence Slit | 1.00 deg |
| Scatter Slit | 1.00 deg |
| Receiving Slit | 0.30 mm |
| Scanning | |
| Scan Range | 3.0-45.0 deg |
| Scan Mode | Continuous |
| Step Size | 0.04° |
| Scan Rate | 2°/min |

5. Karl Fischer Analysis (KF)

Water content was determined by adding solid sample to the instrument with HYDRANAL-Coulomat AD. Micrograms of water were determined by coulometric titration.

6. Moisture-Sorption Analysis

Moisture-sorption experiments were carried out on all samples by first drying the sample at 0% RH and 25° C. until an equilibrium weight was reached or for a maximum of four hours. The sample was then subjected to an isothermal (25° C.) adsorption scan from 10 to 90% RH in steps of 10%. Samples were then allowed to equilibrate to an asymptotic weight at each point for a maximum of four hours. Following adsorption, a desorption scan from 85 to 0% RH (at 25° C.) was run in steps of −10% again allowing a maximum of four hours for equilibration to an asymptotic weight. Samples were dried for one hour at 80° C. and the resulting solids analyzed by XRPD.

7. Nuclear Magnetic Resonance (NMR)

Samples (2 to 10 mg) were dissolved in DMSO-d₆ with 0.05% tetramethylsilane (TMS) for internal reference. ¹H NMR spectra were acquired at 500 MHz using 5 mm broadband observe (¹H—X) Z gradient probe. A 30 degree pulse with 20 ppm spectral width, 1.0 s repetition rate, and 16 to 64 transients were utilized in acquiring the spectra.

### Example 3

### Approximate Solubility of Compound I in Different Solvents

A solubility screen of Compound I was evaluated in 20 different solvents chosen as MeCN, dioxane, acetone, MtBE, EtOH, EtOAc, IPAc, IPA, THF, 2-MeTHF, MEK, DMF, AcOH, MeOH, cyclohexane, heptane, DCM, toluene, NMP, and DMAc, to select appropriate primary and binary solvents for the polymorph screen. One to four mg of the Compound I was charged to 2-dram clear vials equipped with magnetic stir bars and solvent was added in 250-μL portions with heating to 55° C. until complete dissolution was observed or a maximum of 6 mL was added. A summary of the solubility study is presented in Table 10 which shows the solvents that were used and their ability to dissolve the material at room temperature and at 55° C. Solvents and other reagents were of ACS or HPLC grade and were used as received.

### Example 4

### Single-Solvent Crystallizations

Between 5 and 20 mg of Compound I was weighed out into vials and enough solvent was added until the material completely dissolved at elevated temperature. After hot filtration using a 0.45 micron syringe filter the vials were placed in a freezer (−20° C.) for 16 hours in the fast cooling procedure or cooled to room temperature at the rate of 20° C./hour and stirred at this temperature for 16 h in the slow cooling procedure. Table 12 summarizes the experiments.

### Example 5

### Binary-Solvent Crystallizations

Approximately ˜20 mg of Compound I was weighed into vials and solvent was added until the material completely dissolved at elevated temperature. After hot filtration using a 0.45 micron syringe filter the anti-solvent was added portion wise until the solution turned turbid or the vial was full. Then the vials were placed in a freezer (−20° C.) for 16 hours. The samples were evaporated down to dryness using a gentle stream of nitrogen as no precipitation had occurred. All solids from evaporation were dried in vacuo at room temperature and 30 inches Hg for 16 hours. After drying the sample will be analyzed for form by XRPD. Table 13 provides a summary of the experiments.

### Example 6

### Free Basing Experiments of the HCl Salt of Compound I

To obtain Compound I for a polymorph screen, the HCl salt of Compound I (2.52 g) was dissolved with stirring in 100 mL of MeCN: water (1:1, v/v). This mixture was filtered to remove a small amount of undissolved particles. To clarify the solution solid NaHCO₃ (1.99 g, 5.0 equiv.) was added in one portion followed by additional stirring at ambient temperature for 15 minutes. The suspension was concentrated to about half volume by rotary evaporation, and the resultant solution filtered and washed with water (2×25 mL). The filter cake was dried at ambient temperature under vacuum (30 inches Hg) for 24 hours to afford 2.12 g of Compound I (94.3% yield).

Additional freebasing experiments of Compound I were investigated using various solvents in an effort to achieve a reactive crystallization. The HCl salt of Compound I went into EtOH:water and MeOH:water, but it was observed to rapidly precipitate before the addition of the base. Sodium carbonate was added as a 1M solution in water. The reaction mixtures was stirred for 15 minutes after the addition of base and filtered. After filtration the material was dried in vacuo at ambient temperature and 30 inches of Hg. Table 14 shows the amounts of solvent used for these experiments as well as the XRPD results.

### Example 8

### Scale-Up Preparation of Form A

Four separate experiments were conducted, all led to Form A. Table 19 summarizes the details of the experiment.

Experiment 1

HCl salt of Compound I (2.52 g) was charged into a 250 mL-3N RBF equipped with magnetic stir bar followed by the addition of MeCN and water (50 mL each, ˜20 v/v) in one portion at ambient temperature. The resultant solution was polish filtered through Whatman #1 filter paper followed by the addition of NaHCO₃ (1.99 g, 5.0 equiv.) and further stirring at ambient temperature for 15 min. The resultant slurry was concentrated under reduced pressure followed by filtration of the solids. The filter cake was then dried at ambient temperature for 24 h to afford Form A of Compound I (2.2 g, 94.3% yield) by XRPD. Form A was found.

Experiment 2

Compound I (Form A, 0.244 g) was charged into a 20 mL-scintillation vial equipped with magnetic stir bar followed by the addition of IPA (8 mL, 32 vol.) in one portion at 40° C. The resultant mixture was then slurried at 40° C. for one week. Solids were isolated by filtration and dried under vacuum at ambient temperature for 16 h before being submitted for analysis by XRPD. Form A was found.

Experiment 3

Compound I (Form A, 0.313 g) was charged into a 20 mL-scintillation vial equipped with magnetic stir bar followed by the addition of dioxane (10 mL, 32 vol.) in one portion at 40° C. The resultant mixture was then slurried at 40° C. for one week. Solids were isolated by filtration and dried under vacuum at ambient temperature for 16 h before being submitted for analysis by XRPD. Form A was found.

Experiment D

Compound I (Form A, 0.309 g) was charged into a 20 mL-scintillation vial equipped with magnetic stir bar and thermocouple followed by the addition of acetone (10 mL, 32 vol.) in one portion at 40° C. The resultant mixture was then slurried at 40° C. for one week. Solids were isolated by filtration and dried under vacuum at ambient temperature for 16 h before being submitted for analysis by XRPD. Form A was found.

### Example 9

### Slurry Experiments

50 to 60 mg of Form A of Compound I was weighed out into 2 dram amber vials and the material was slurried in 2 mL of a solvent. The slurry experiments were carried out at either ambient temperature or at 40° C. Samples were obtained from slurries after one day, one week, and two weeks. Tables 15 and 16 detail the results of the slurry experiments.

The results showed that Form A and/or Form C were isolated from most crystallization, with the Form A in dioxane slurry showing a pattern consistent with a mixture of Form A and Form D. Form D was observed previously from dioxane slurries at ambient temperature and at 40° C. These results are consistent with Form A being preferentially isolated from MeCN and Form C from THF or MeOH.

### Example 10

### Solubility Study

Solubility was determined for Form A of Compound I by suspending the material in de-ionized water and 20 mM phosphate buffer at pH 3.2 and ambient temperature. Approximately 40 mg of Form A of Compound I was charged to 2-dram amber vials equipped with magnetic stir bars, followed by the addition of DI water or phosphate buffer (2.0 mL) in one portion. The samples were allowed to equilibrate with stirring for 16 h, followed by centrifugation. An aliquot of the supernatant liquid was analyzed by HPLC, and the solids isolated by filtration and dried under vacuum at ambient temperature for analysis by XRPD. Samples were allowed to stir for an additional 6 days and concentration determined by HPLC. Table 10 summarizes the experimental details and results.

TABLE 9
Analytical results summary for Forms A through G of Compound I.

| | | | KF | TGA | |
|---|---|---|---|---|---|
| Form | | DSC | (wt % | (wt % | |
| (XRPD) | Conditions | (° C.) | water) | lost) | ¹H NMR |
| A | Slurry in | 67, 229, 278, | 5.0% | 2.2% | Consistent |
| | MeCN | 280\hat{ }, 293 | | | |
| | (RT) | | | | |
| B | Several w/ | N/A | N/A | N/A | N/A |
| | counter-ion | | | | |
| C | FB in MeOH | 68, 83\hat{ }, 88, | 3.0% | 3.2% | Consistent |
| | | 241, 277\hat{ }, | | | |
| | | 291 | | | |
| D | Slurry in | 282, 284\hat{ }, | 1.4% | 0.9% | Consistent |
| | dioxane | 291 | | | |
| | (RT, 40° C.) | | | | |
| E | Slurry in | 276, 278\hat{ }, | 0.8% | 3.0% | Consistent |
| | acetone | 291 | | | |
| | (40° C.) | | | | |
| F | Elevated | 149, 228, 289, | N/A* | N/A* | Consistent |
| | temperatures | 299 | | | |
| G | Elevated | 299 | N/A* | N/A* | Consistent |
| | temperatures | | | | |

\hat{ }indicates exothermic event
*These forms were isolated by heat-cool experiments in DSC.

TABLE 10
Solubility of Compound I Form A.

| | | Solubility | | Solubility | |
|---|---|---|---|---|---|
| | | mg/mL | XRPD | mg/mL | XRPD |
| Media | pH (initial) | (1 day) | (1 day) | (pH, 1 week) | (1 week) |
| DI water | 6.9 | <0.1 | Form A | <0.1 (7.2) | Form A |
| Phosphate | 3.2 | <0.1 | Form A | <0.1 (3.4) | Form A |
| buffer | | | | | |

TABLE 11
Initial solubility of Compound I

| | Material | Solvent | | | | |
|---|---|---|---|---|---|---|
| | Amt | Amt | Conc. | | | ICH |
| Solvent | (mg) | (mL) | (mg/mL) | Temp | Soluble | Class |
| MeCN | 1.7 | 5.75 | 0.30 | 55 | Yes | II |
| Dioxane | 1.7 | 5.75 | — | — | Partial | II |
| Acetone | 1.8 | 5.75 | — | — | Partial | III |
| MTBE | 1.3 | 5.75 | — | — | No | III |
| EtOH | 1.6 | 4.75 | 0.34 | 55 | Yes | III |
| EtOAc | 2.2 | 5.75 | — | — | No | III |
| IPAC | 2.6 | 5.75 | — | — | No | III |
| IPA | 1.2 | 5.75 | — | — | Partial | III |
| THF | 1.3 | 3.75 | 0.35 | 55 | Yes | II |
| 2-Me—THF | 1.6 | 5.75 | — | — | No | N/A |
| MEK | 1.9 | 5.75 | — | — | Partial | III |
| DMF | 1.8 | 0.50 | >3.60 | RT | Yes | II |
| AcOH | 2.1 | 0.75 | 2.80 | RT | Yes | III |
| MeOH | 1.7 | 2.00 | 0.85 | 55 | Yes | II |
| c-Hexane | 1.8 | 3.50 | — | — | No | II |
| Heptane | 1.9 | 3.50 | — | — | No | III |
| DCM | 2.6 | 4.50 | — | — | No | II |
| Toluene | 1.8 | 3.50 | — | — | No | II |
| NMP | 2.4 | 0.50 | 4.80 | 55 | Yes | II |
| DMA | 2.8 | 0.50 | 5.60 | 55 | Yes | II |

c-hexane = cyclohexane
— = not dissolved
“Partial” indicates some material was observed to dissolve

TABLE 12
Single solvent crystallizations of Compound I
using fast and slow cooling procedure

| Material | | Solvent | Cooling | | | Form |
|---|---|---|---|---|---|---|
| Amt (mg) | Solvent | Amt (mL) | Profile | Filtered | Evap | (XRPD) |
| 5.1 | MeCN | 17.00 | Fast | No | Yes | C |
| 4.9 | EtOH | 6.00 | Fast | No | Yes | C |
| 5.7 | THF | 15.00 | Fast | No | Yes | amorphous |
| 19.0 | DMF | 9.00 | Fast | No | Yes | C |
| 5.3 | MeOH | 15.00 | Fast | No | Yes | C |
| 23.4 | NMP | 8.00 | Fast | No | Yes | C |
| 20.5 | DMA | 8.00 | Fast | No | Yes | C |
| 5.2 | MeCN | 17.00 | Slow | No | Yes | C |
| 4.6 | EtOH | 6.00 | Slow | No | Yes | N/A |
| 5.3 | THF | 15.00 | Slow | No | Yes | amorphous |
| 17.0 | DMF | 9.00 | Slow | No | Yes | C |
| 5.7 | MeOH | 15.00 | Slow | No | Yes | C |
| 18.6 | NMP | 8.00 | Slow | No | Yes | N/A |
| 19.6 | DMA | 8.00 | Slow | No | Yes | C |

N/A indicates sample was not analyzed

TABLE 13
Binary solvent crystallizations of Compound I
using fast cooling procedure and DMF as primary solvent

| Material | Main | Solvent | Anti | Solvent | Cooling | | | Form |
|---|---|---|---|---|---|---|---|---|
| Amt (mg) | Solvent | Amt (mL) | Solvent | Amt (mL) | Profile | Filtered | Evap | (XRPD) |
| 14.4 | DMF | 8.0 | MeCN | 10.0 | Fast | No | Yes | C |
| 14.2 | DMF | 8.0 | EtOAc | 10.0 | Fast | No | Yes | C |
| 15.9 | DMF | 8.0 | IPAC | 10.0 | Fast | No | Yes | C |
| 15.0 | DMF | 8.0 | c-hexane | 10.0 | Fast | No | Yes | C |
| 16.0 | DMF | 8.0 | Heptane | 10.0 | Fast | No | Yes | C |
| 14.0 | DMF | 8.0 | Toluene | 10.0 | Fast | No | Yes | C |
| 13.7 | DMF | 8.0 | Me—THF | 10.0 | Fast | No | Yes | C |
| 13.2 | DMF | 8.0 | water | 10.0 | Fast | No | Yes | C |

TABLE 14
Reactive crystallization of Compound I via neutralization
of Compound I•HCl in various solvents

| | | | | 1M solution | |
|---|---|---|---|---|---|
| Material | | Solvent Amt | Water Amt | of NaCO₃ | Form |
| Amt (mg) | Solvent | (μL) | (μL) | (μL) | (XRPD) |
| 29.5 | MeCN | 500.0 | 225.0 | 275.0 | C |
| 29.5 | EtOH | 500.0 | 225.0 | 275.0 | C |
| 29.3 | MeOH | 500.0 | 230.0 | 270.0 | C |
| 26.3 | THF | 500.0 | 255.0 | 245.0 | C |
| 27.0 | Dioxane | 500.0 | 250.0 | 250.0 | C |
| 28.7 | IPA | 500.0 | 235.0 | 265.0 | C |

TABLE 15
Slurry experiments of Compound I

| Material | Slurry | Solvent | Temp | XRPD Results | | |
|---|---|---|---|---|---|---|
| Amt (mg) | Solvent | Amt (mL) | ° C. | Day 1* | Week 1* | Week 2 |
| 50.4 | MeCN | 2.0 | Ambient | C | A | A |
| 51.1 | dioxane | 2.0 | Ambient | A | D | D |
| 59.6 | Acetone | 2.0 | Ambient | amorphous | A | A |
| 50.9 | EtOH | 2.0 | Ambient | C | C | C |
| 51.3 | IPA | 2.0 | Ambient | C | C | C |
| 50.4 | THF | 2.0 | Ambient | amorphous | C | C |
| 56.2 | MEK | 2.0 | Ambient | amorphous | C | C |
| 53.5 | MeOH | 2.0 | Ambient | C | C | C |
| 60.2 | MeCN | 2.0 | 40 | C | C | C |
| 52.0 | dioxane | 2.0 | 40 | A | D | D |
| 63.8 | Acetone | 2.0 | 40 | C | E | E |
| 56.1 | EtOH | 2.0 | 40 | C | C | C |
| 52.9 | IPA | 2.0 | 40 | C | C | C |
| 57.8 | THF | 2.0 | 40 | C | C | C |
| 49.7 | MEK | 2.0 | 40 | A | A | A |
| 60.8 | MeOH | 2.0 | 40 | C | C | C |
| 50.0 | water | 2.0 | Ambient | amorphous | amorphous | C |
| 49.9 | water | 2.0 | 40 | amorphous | amorphous | C |

*These samples provided limited material and definitive determination of form was not possible in all experiments.

TABLE 16
Additional slurry experiments of Compound I
Forms A and C at ambient temperature.

| Initial | Material | Slurry | Solvent Amt | | Final Form |
|---|---|---|---|---|---|
| form | Amt (mg) | Solvent | (mL) | Temp | (1 week) |
| A | 40.5 | MeCN | 1.6 | Ambient | A |
| | 41.5 | dioxane | 1.6 | Ambient | A + D |
| | 43.2 | Acetone | 1.6 | Ambient | A |
| | 42.1 | EtOH | 1.6 | Ambient | A |
| | 42.6 | IPA | 1.6 | Ambient | A |
| | 41.2 | THF | 1.6 | Ambient | C |
| | 39.8 | MEK | 1.6 | Ambient | A + C |
| | 39.8 | MeOH | 1.6 | Ambient | C |
| | 40.0 | water | 1.6 | Ambient | A + C |
| C | 37.2 | MeCN | 1.6 | Ambient | A |
| | 37.2 | dioxane | 1.6 | Ambient | C |
| | 36.8 | Acetone | 1.6 | Ambient | C |
| | 38.0 | EtOH | 1.6 | Ambient | C |
| | 39.2 | IPA | 1.6 | Ambient | C |
| | 37.4 | THF | 1.6 | Ambient | C |
| | 37.6 | MEK | 1.6 | Ambient | A + C |
| | 37.4 | MeOH | 1.6 | Ambient | C |
| | 39.0 | water | 1.6 | Ambient | C |

TABLE 17
DSC experiments on Compound I Form A to determine
if isolation of high-melt forms was possible.

| DSC Segments | DSC events observed on final segment (° C.) |
|---|---|
| 1. 30-350° C. @ | 57, 230, 275, 278\hat{ }, 290, 300 |
| 10° C./min | |
| 1. 30-80° C. @ | 228, 275\hat{ }, 277\hat{ }, 290, 299 |
| 10° C./min | |
| 2. Hold 80° C. | |
| for 5 min | |
| 3. 80-30° C. @ | |
| −5° C./min | |
| 4. 30-350° C. @ | |
| 10° C./min | |
| 1. 30-225° C. @ | 276\hat{ }, 281, 289, 299 |
| 10° C./min | |
| 2. Hold 225° C. | |
| for 5 min | |
| 3. 225-30° C. @ | |
| −5° C./min | |
| 4. 30-350° C. @ | |
| 10° C./min | |
| 1. 30-240° C. @ | 277\hat{ }, 282, 289, 299 |
| 10° C./min | |
| 2. Hold 240° C. | |
| for 5 min | |
| 3. 240-30° C. @ | |
| −5° C./min | |
| 4. 30-350° C. @ | |
| 10° C./min | |
| 1. 30-270° C. @ | 289, 299* |
| 10° C./min | |
| 2. Hold 270° C. | |
| for 5 min | |
| 3. 270-30° C. @ | |
| −5° C./min | |
| 4. 30-350° C. @ | |
| 10° C./min | |
| 1. 30-280° C. @ | 289, 299* |
| 10° C./min | |
| 2. Hold 280° C. | |
| for 5 min | |
| 3. 280-30° C. @ | |
| −5° C./min | |
| 4. 30-350° C. @ | |
| 10° C./min | |
| 1. 30-290° C. @ | 299 |
| 10° C./min | |
| 2. Hold 290° C. | |
| for 5 min | |
| 3. 290-30° C. @ | |
| −5° C./min | |
| 4. 30-350° C. @ | |
| 10° C./min | |

\hat{ }indicates exothermic event
*Difference in intensity ratio of the two events was observed with the intensity for the event at 289 being much greater than the intensity for the event at 299

TABLE 18
DSC experiments on Compound I Form A to isolate high-melt forms.

| DSC Segments | XRPD | DSC (° C.) | ¹H NMR |
|---|---|---|---|
| 1. 30-280° C. @ 10° C./min | Form F | 289, 299* | No degradation |
| 2. Hold 280° C. for 5 min | | | observed |
| 3. 280-30° C. @ - | | | |
| 5° C./min | | | |
| 1. 30-290° C. @ 10° C./min | Form G | 299 | No degradation |
| 2. Hold 290° C. for 5 min | | | observed |
| 3. 290-30° C. @ - | | | |
| 5° C./min | | | |

*Additional small peaks observed at 149 and 228° C.

TABLE 19
Scale up preparation of Compound I Forms A, C, D, and E.

| Description | | | | | |
|---|---|---|---|---|---|
| of starting | Material | | Temp | Intended | Isolated |
| material | Amt (mg) | Solvent | (° C.) | Form | Form |
| Compound | 2 g | MeCN/water | RT | A | A |
| I•HCl | | | | | |
| Form A | 244 | IPA | 40 | C | A |
| Form A | 313 | dioxane | 40 | D | A |
| Form A | 309 | acetone | 40 | E | A |

TABLE 20
Humidity chamber study of Compound I Form A.

| Form | KF | Conditions | Result Form | Result KF |
|---|---|---|---|---|
| (initial) | (initial) | (% RH) | (1 week) | (1 week) |
| A | 4.6 | 0% | A | 4.4 |

1. (canceled)

2. The polymorphic form of Compound I having the formula 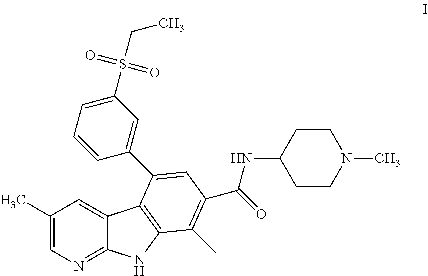
wherein the polymorphic form is Form A which is a dihydrate having an X-ray powder diffraction pattern (CuKα) comprising significant diffraction peaks at about 5.4, 17.3 and 20.2 degrees 2-theta (°2θ).

3. The polymorphic form of claim 2, wherein the X-ray powder diffraction pattern (CuKα) further comprises significant diffraction peaks at about 16.7, 20.7 and 25.2 °2θ.

4-78. (canceled)
